(19) United States
(12) Patent Application Publication
(10) Pub. No.: US 2018/0127403 A1
(43) Pub. Date: May 10, 2018

(54) N-ARYL-HYDROACRIDINES AS LIGHT EMITTING ELEMENTS FOR ELECTROLUMINESCENT DEVICES

(71) Applicants: Dow Global Technologies LLC, Midland, MI (US); Rohm and Haas Electronic Materials LLC, Marlborough, MA (US)

(72) Inventors: Nolan T. McDougal, Houston, TX (US); David S. Laitar, Midland, MI (US); Sukrit Mukhopadhyay, Midland, MI (US); Thomas P. Clark, Midland, MI (US); David D. Devore, Midland, MI (US); Kaitlyn Gray, Indianapolis, IN (US); Aaron A. Rachford, South Grafton, MA (US); Robert DJ Froese, Midland, MI (US)

(21) Appl. No.: 15/570,578

(22) PCT Filed: Jul. 7, 2016
(86) PCT No.: PCT/US16/41239

§ 371 Date: Oct. 30, 2017

Related U.S. Application Data
(60) Provisional application No. 62/193,828, filed on Jul. 17, 2015.

(51) Int. Cl. C07D 413/14; C07D 413/04; C07D 401/14; C07D 401/04; C09K 11/06; H01L 51/00

(52) U.S. Cl. CPC C07D 413/14; C07D 413/04; C07D 401/14; H01L 51/5012; C09K 11/06; H01L 51/0072; C07D 401/04

(57) ABSTRACT

A compound having a tricyclic nucleus and having formula (I)

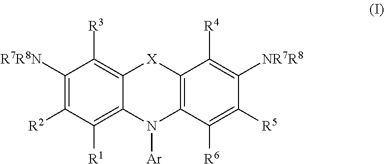

wherein Ar is C₃-C₂₅ aryl in which at least one aromatic ring atom is nitrogen; X is O, S or CR⁹R¹⁰; R¹, R², R³, R⁴, R⁵ and R⁶ independently are hydrogen, deuterium, C₁-C₁₂ alkyl, C₄-C₁₂ aryl or C₂-C₁₂ alkenyl; R⁷ and R⁸ independently are hydrogen, deuterium, C₁-C₁₂ alkyl, C₄-C₁₂ aryl or C₂-C₁₂ alkenyl or R⁷ and R⁸ groups attached to a nitrogen atom are joined by a single bond, O, S or CR¹¹R¹² to form a single nitrogen-containing substituent; R⁹ and R¹⁰ independently are hydrogen, deuterium, C₁-C₁₂ alkyl, C₄-C₁₂ aryl or C₂-C₁₂ alkenyl; and R¹¹ and R¹² independently are hydrogen, deuterium, C₁-C₁₂ alkyl, C₄-C₁₂ aryl or C₂-C₁₂ alkenyl; provided that Ar does not contain an aromatic ring attached to the tricyclic nucleus which contains more than two aromatic ring nitrogen atoms.

This invention relates to new N-aryl hydroacridine compounds useful as emitters in organic light-emitting diode (OLED) displays.

N-aryl hydroacridine compounds potentially useful in OLED displays are known. For example, WO2006/033563 discloses a compound having the structure

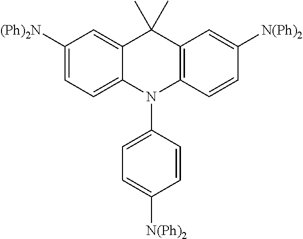

However, this reference does not disclose the compounds claimed herein. There is a need for emitters having a higher efficiency. The problem addressed by this invention is to find additional emitter compounds useful in OLED displays, and thermally activated delayed fluorescent (TADF) emitters in particular.

### STATEMENT OF INVENTION

The present invention provides a compound having a tricyclic nucleus and having formula (I)

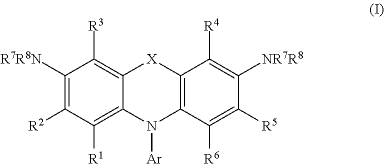

wherein Ar is C₃-C₂₅ aryl in which at least one aromatic ring atom is nitrogen; X is O, S or CR⁹R¹⁰; R¹, R², R³, R⁴, R⁵ and R⁶ independently are hydrogen, deuterium, C₁-C₁₂ alkyl, C₄-C₁₂ aryl or C₂-C₁₂ alkenyl; R⁷ and R⁸ independently are hydrogen, deuterium, C₁-C₁₂ alkyl, C₄-C₂₀ aryl or C₂-C₁₂ alkenyl or R⁷ and R⁸ groups attached to a nitrogen atom are joined by a single bond, O, S or CR¹¹R¹² to form a single nitrogen-containing substituent; R⁹ and R¹⁰ independently are hydrogen, deuterium, C₁-C₁₂ alkyl, C₄-C₁₂ aryl or C₂-C₁₂ alkenyl; and R¹¹ and R¹² independently are hydrogen, deuterium, C₁-C₁₂ alkyl, C₄-C₁₂ aryl or C₂-C₁₂ alkenyl; provided that Ar does not contain an aromatic ring attached to the tricyclic nucleus which contains more than two aromatic ring nitrogen atoms.

The present invention further provides a light-emitting device comprising at least one compound having formula (I).

### DETAILED DESCRIPTION

Percentages are weight percentages (wt %) and temperatures are in ° C., unless specified otherwise. Experimental work was carried out at room temperature (“rt”; 20-25° C.), unless otherwise specified. “Dopant” refers to a material that undergoes radiative emission from an excited state. This excited state can be generated by application of electrical current in an electroluminescent device and is either singlet or triplet in character. The term “fluorescent emission,” as used herein, refers to radiative emission from a singlet excited state. The term “phosphorescent emission,” as used herein, refers to radiative emission from a triplet excited state. For a dopant that undergoes primarily fluorescent emission, the term “triplet harvesting,” as used herein, refers to the ability to also harvest triplet excitons. The term “thermally activated delayed fluorescence (TADF),” as used herein, refers to fluorescent emission utilizing triplet harvesting, enabled by a thermally accessible singlet excited state. “Host” and like terms refer to a material that is doped with a dopant. The opto-electrical properties of the host material may differ based on which type of dopant (Phosphorescent or Fluorescent) is used. For Fluorescent dopants, the assisting host materials should have good spectral overlap between adsorption of the dopant and emission of the host to induce good Forster transfer to dopants. For Phosphorescent dopants and TADF dopants, the assisting host materials should have high triplet energies to confine triplets on the dopant.

A “tricyclic nucleus” is a system of three fused rings to which substituents are attached. The tricyclic nucleus of the compound of formula (I) is as shown below

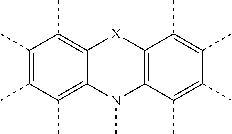

with dashed lines indicating attachments to substituents. An “aromatic ring atom” is an atom which is part of an aromatic ring; for example, the carbon atoms in the tricyclic nucleus shown above are aromatic ring atoms, but X and N are not. An “alkyl” group is a substituted or unsubstituted hydrocarbyl group having from one to twelve carbon atoms in a linear, branched or cyclic arrangement. Preferably, alkyl groups are unsubstituted. Preferably, alkyl groups are linear or branched, i.e., acyclic. Preferably, each alkyl substituent is not a mixture of different alkyl groups, i.e., it comprises at least 98% of one particular alkyl group. An “alkenyl” group is an alkyl group having at least one carbon-carbon double bond, preferably one or two, preferably one. An “aryl” group is a substituent group containing at least one aromatic ring. In addition to carbocyclic aromatic rings, aryl groups may include aromatic rings containing heteroatoms, e.g., pyridyl, pyrimidinyl, pyrrolyl and pyrazinyl; and/or alicyclic rings. Aryl groups may be substituted by one or more C₁-C₈ alkyl or C₂-C₈ alkenyl substituents, preferably C₁-C₆ alkyl, preferably C₁-C₄ alkyl; in a preferred embodiment, aryl groups are unsubstituted or substituted only by deuterium or one to three methyl or ethyl groups, preferably one or two methyl groups. Carbon numbers for aryl groups include carbon atoms in substituents. Where hydrogen atoms are present in the compounds of this invention, they can be partially or completely replaced by deuterium atoms, although hydrogen (i.e., the naturally occurring isotopic mixture) is preferred. Preferably, the compounds of this invention are neutral, i.e., they have no overall charge.

Preferably, the compounds of this invention have a molecular weight from 400 to 900, preferably from 440 to 850, preferably from 500 to 800.

Preferably, R¹, R², R³, R⁴, R⁵ and R⁶ independently are hydrogen, deuterium, C₁-C₈ alkyl, C₄-C₁₂ aryl or C₂-C₈ alkenyl; preferably hydrogen, deuterium or C₁-C₄ alkyl; preferably hydrogen, deuterium or C₁-C₂ alkyl; preferably hydrogen or deuterium. Preferably, R⁹ and R¹⁰ independently are hydrogen, deuterium, C₁-C₈ alkyl, C₄-C₁₂ aryl or C₂-C₈ alkenyl; preferably hydrogen, deuterium, C₆-C₁₀ aryl or C₁-C₄ alkyl; preferably hydrogen, deuterium or C₁-C₂ alkyl; preferably methyl or ethyl. Preferably, R¹¹ and R¹² independently are hydrogen, deuterium, C₁-C₈ alkyl, C₄-C₁₂ aryl or C₂-C₈ alkenyl; preferably hydrogen, deuterium or C₁-C₄ alkyl; preferably hydrogen, deuterium or C₁-C₂ alkyl; preferably methyl or ethyl. Preferably, R⁷ and R⁸ independently are C₁-C₁₂ alkyl, C₄-C₁₈ aryl or C₂-C₁₂ alkenyl; preferably C₄-C₁₅ aryl, preferably C₄-C₁₀ aryl.

In the compounds of this invention, Ar does not contain an aromatic ring attached to the tricyclic nucleus (i.e., to the ring nitrogen atom of the tricyclic nucleus) which contains more than two aromatic ring nitrogen atoms. However, Ar may contain one or more aromatic rings which do contain more than two aromatic ring nitrogen atoms, provided that these aromatic rings are not attached directly to the nitrogen atom of the tricyclic nucleus. The number of carbon atoms indicated for Ar includes any alkyl substituents present on one or more rings within Ar. Preferably, Ar is C₆-C₂₅ aryl, preferably C₆-C₂₀ aryl, preferably C₉-C₂₀ aryl. Preferably, Ar comprises two or three aromatic rings; preferably two. Preferably, Ar has from one to six aromatic ring atoms which are nitrogen, preferably from one to five, preferably from one to four, preferably from one to three, preferably one or two. Preferably, no aromatic ring in Ar contains more than two aromatic ring nitrogen atoms, preferably no more than one aromatic nitrogen ring atom. Preferably, X is O or CR⁹R¹⁰.

In a preferred embodiment, R⁷ and R⁸ groups attached to the same nitrogen atom and which are aryl groups may join to form a single substituent, e.g.,

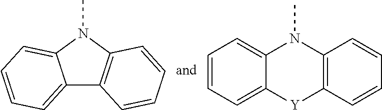

wherein Y is O, S or CR¹¹R¹²; with a dashed line showing the point of attachment to the tricyclic nucleus.

In a preferred embodiment, the compounds of this invention have formula (II)

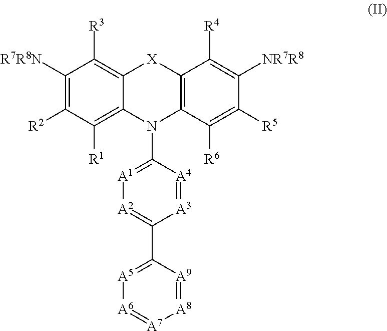

wherein A¹, A², A³, A⁴, A⁵, A⁶, A⁷, A⁸ and A⁹ independently are N or CR, wherein R is the same or different in different A groups and may be hydrogen, deuterium, C₁-C₁₂ alkyl, C₄-C₁₂ aryl or C₂-C₁₂ alkenyl; provided that at least one of A¹, A², A³, A⁴, A⁵, A⁶, A⁷, A⁸ and A⁹ is N and no more than two of A¹, A², A³ and A⁴ are N. Other substituents are as defined previously. Preferably, R is hydrogen, deuterium, C₁-C₆ alkyl, C₄-C₁₀ aryl or C₂-C₆ alkenyl; preferably hydrogen, deuterium, C₁-C₄ alkyl or C₂-C₄ alkenyl; preferably hydrogen, deuterium, methyl or ethyl. Preferably, no more than six of A¹, A², A³, A⁴, A⁵, A⁶, A⁷, A⁸ and A⁹ are nitrogen, preferably no more than five, preferably no more than four, preferably no more than three, preferably no more than two. Preferably, at least three of A¹, A², A³, A⁴, A⁵, A⁶, A⁷, A⁸ and A⁹ are CH or CD, preferably at least four. Preferably, no more than two of A⁵, A⁶, A⁷, A⁸ and A⁹ are N. Preferably, no more than one of A¹, A², A³ and A⁴ is N.

In a preferred embodiment, the compounds of this invention have formula (III)

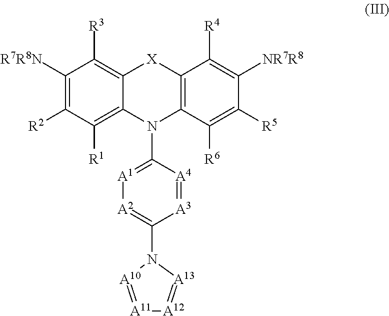

wherein A¹⁰, A¹¹, A¹² and A¹³ independently are N or CR; at least one of A¹, A², A³, A⁴, A¹⁰, A¹¹, A¹² and A¹³ is N; no more than two of A¹, A², A³ and A⁴ are N and other substituents are as defined previously. Preferably, no more than five of A¹, A², A³, A⁴, A¹⁰, A¹¹, A¹² and A¹³ are N, preferably no more than four, preferably no more than three. Preferably, at least two of A¹, A², A³, A⁴, A¹⁰, A¹¹, A¹² and A¹³ are CH or CD, preferably at least three.

The following structures represent preferred embodiments of the invention:

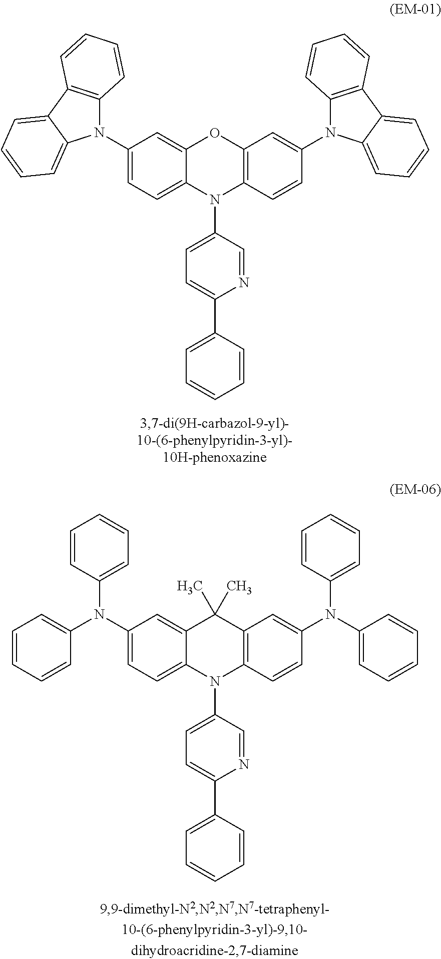 [SMILES: c1ccc(-c2ccc(N3c4ccc(-n5c6ccccc6c6ccccc65)cc4Oc4cc(-n5c6ccccc6c6ccccc65)ccc43)cn2)cc1 ; CC1(C)c2cc(N(c3ccccc3)c3ccccc3)ccc2N(c2ccc(-c3ccccc3)nc2)c2ccc(N(c3ccccc3)c3ccccc3)cc21]

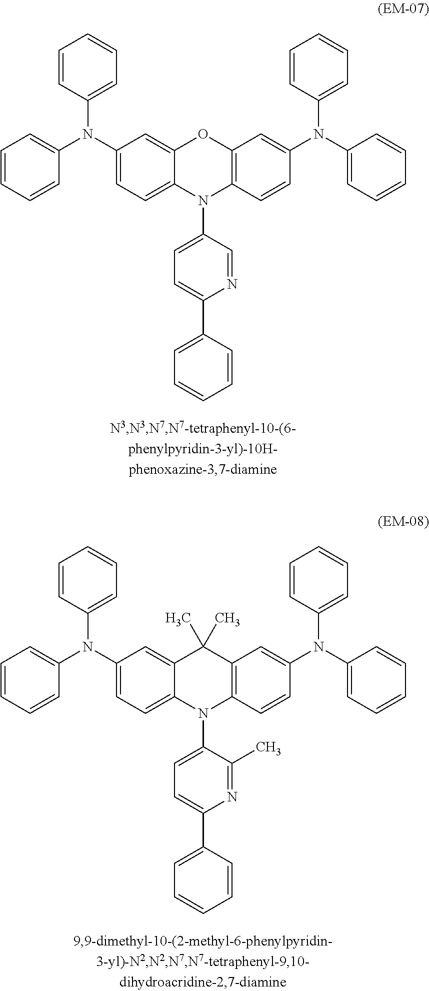 [SMILES: c1ccc(-c2ccc(N3c4ccc(N(c5ccccc5)c5ccccc5)cc4Oc4cc(N(c5ccccc5)c5ccccc5)ccc43)cn2)cc1 ; Cc1nc(-c2ccccc2)ccc1N1c2ccc(N(c3ccccc3)c3ccccc3)cc2C(C)(C)c2cc(N(c3ccccc3)c3ccccc3)ccc21]

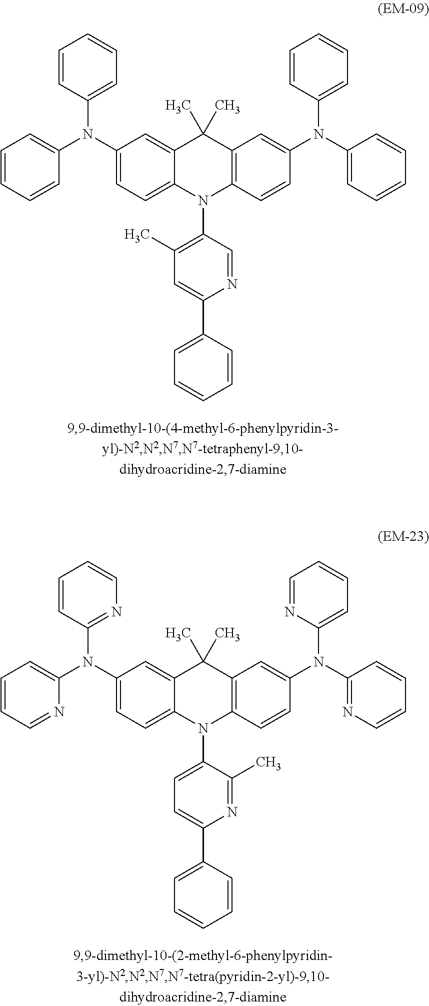 [SMILES: Cc1cc(-c2ccccc2)ncc1N1c2ccc(N(c3ccccc3)c3ccccc3)cc2C(C)(C)c2cc(N(c3ccccc3)c3ccccc3)ccc21 ; Cc1nc(-c2ccccc2)ccc1N1c2ccc(N(c3ccccn3)c3ccccn3)cc2C(C)(C)c2cc(N(c3ccccn3)c3ccccn3)ccc21]

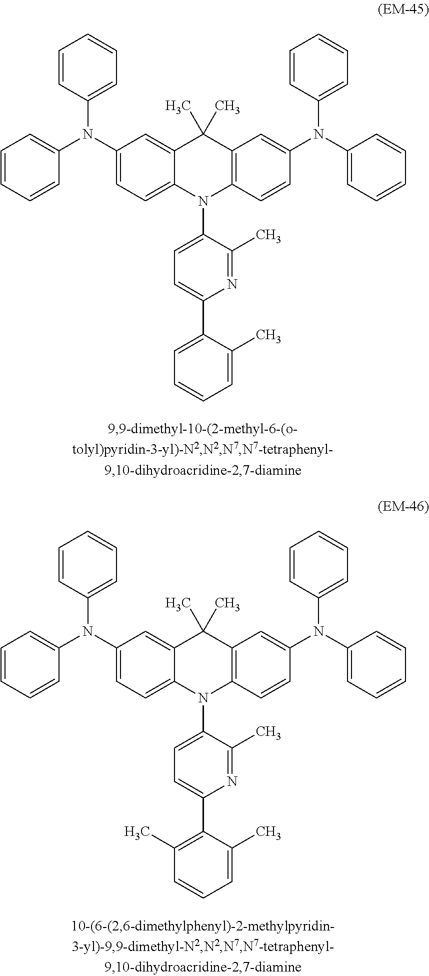 [SMILES: Cc1ccccc1-c1ccc(N2c3ccc(N(c4ccccc4)c4ccccc4)cc3C(C)(C)c3cc(N(c4ccccc4)c4ccccc4)ccc32)c(C)n1 ; Cc1cccc(C)c1-c1ccc(N2c3ccc(N(c4ccccc4)c4ccccc4)cc3C(C)(C)c3cc(N(c4ccccc4)c4ccccc4)ccc32)c(C)n1]

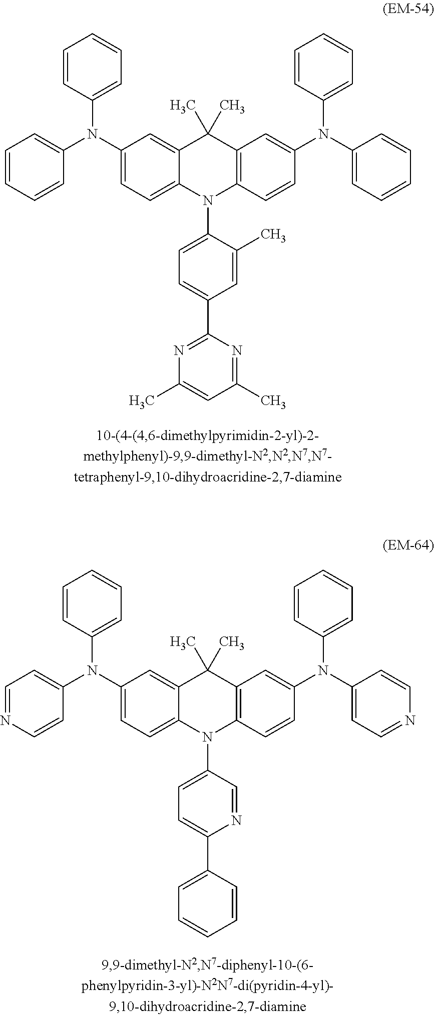 [SMILES: Cc1cc(C)nc(-c2ccc(N3c4ccc(N(c5ccccc5)c5ccccc5)cc4C(C)(C)c4cc(N(c5ccccc5)c5ccccc5)ccc43)c(C)c2)n1 ; CC1(C)c2cc(N(c3ccccc3)c3ccncc3)ccc2N(c2ccc(-c3ccccc3)nc2)c2ccc(N(c3ccccc3)c3ccncc3)cc21]

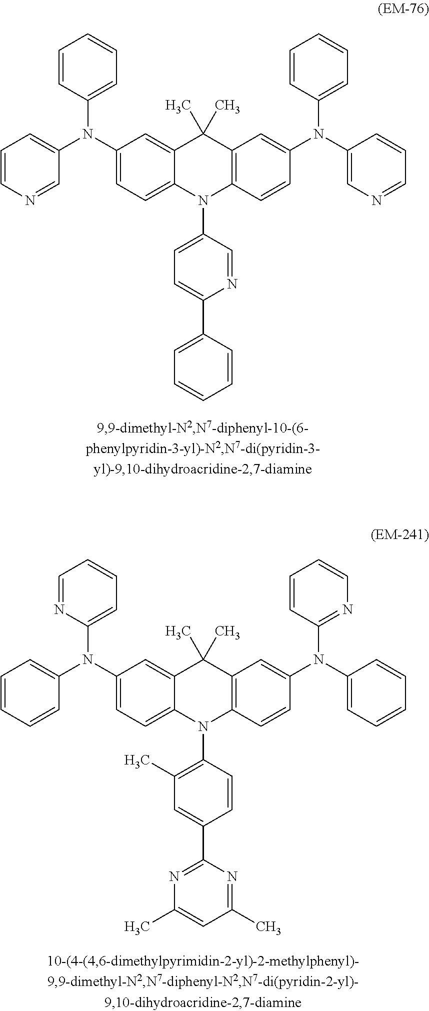 [SMILES: CC1(C)c2cc(N(c3ccccc3)c3cccnc3)ccc2N(c2ccc(-c3ccccc3)nc2)c2ccc(N(c3ccccc3)c3cccnc3)cc21 ; Cc1cc(C)nc(-c2ccc(N3c4ccc(N(c5ccccc5)c5ccccn5)cc4C(C)(C)c4cc(N(c5ccccc5)c5ccccn5)ccc43)c(C)c2)n1]

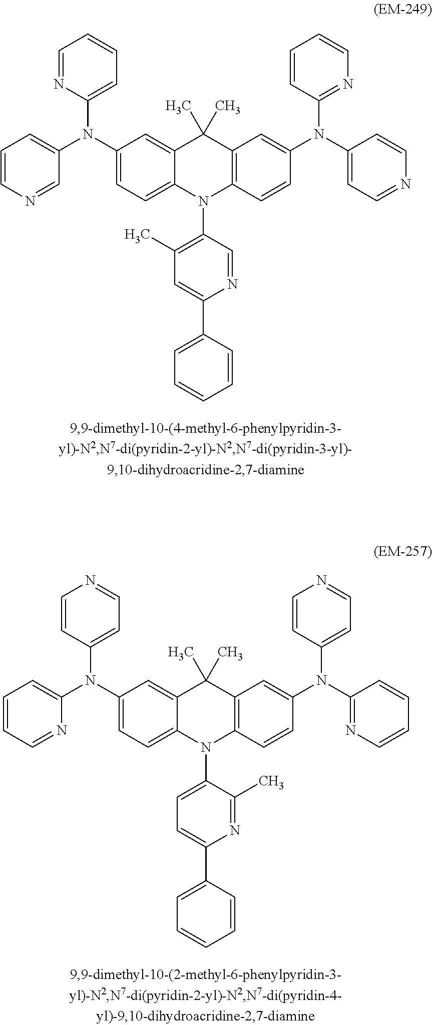 [SMILES: Cc1cc(-c2ccccc2)ncc1N1c2ccc(N(c3ccncc3)c3ccccn3)cc2C(C)(C)c2cc(N(c3cccnc3)c3ccccn3)ccc21 ; Cc1nc(-c2ccccc2)ccc1N1c2ccc(N(c3ccncc3)c3ccccn3)cc2C(C)(C)c2cc(N(c3ccncc3)c3ccccn3)ccc21]

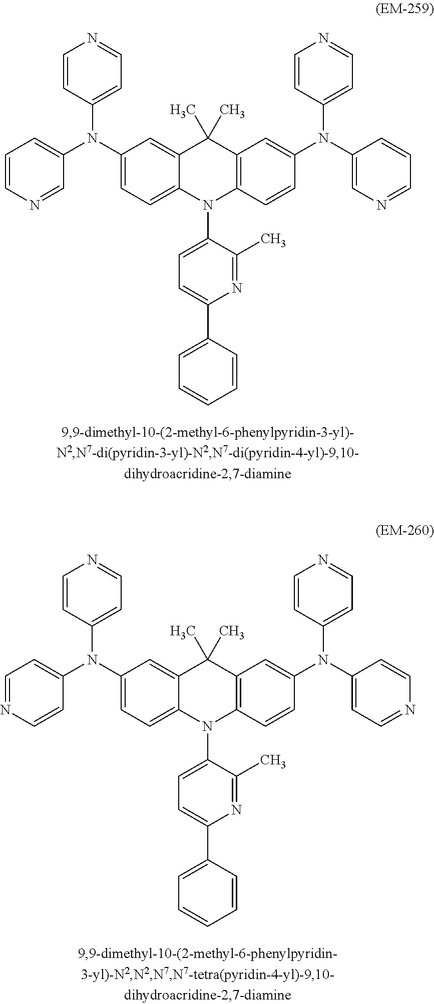 [SMILES: Cc1nc(-c2ccccc2)ccc1N1c2ccc(N(c3ccncc3)c3cccnc3)cc2C(C)(C)c2cc(N(c3ccncc3)c3cccnc3)ccc21 ; Cc1nc(-c2ccccc2)ccc1N1c2ccc(N(c3ccncc3)c3ccncc3)cc2C(C)(C)c2cc(N(c3ccncc3)c3ccncc3)ccc21]

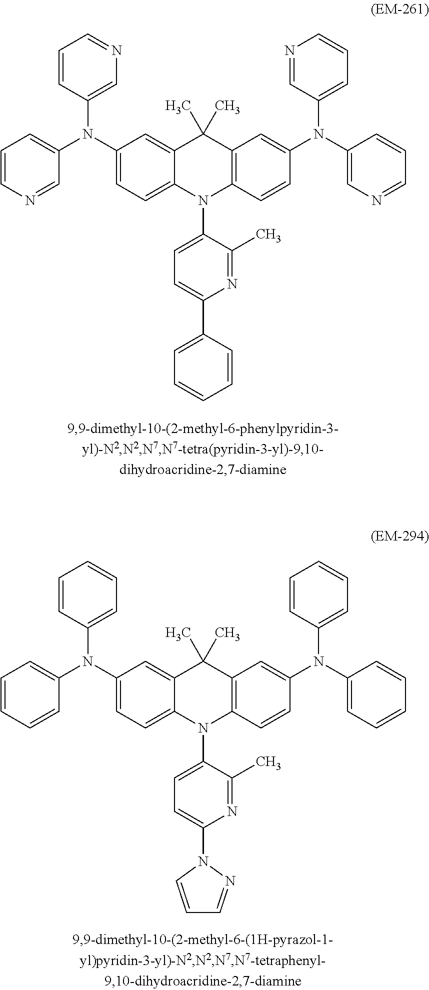 [SMILES: Cc1nc(-c2ccccc2)ccc1N1c2ccc(N(c3cccnc3)c3cccnc3)cc2C(C)(C)c2cc(N(c3cccnc3)c3cccnc3)ccc21 ; Cc1nc(-n2cccn2)ccc1N1c2ccc(N(c3ccccc3)c3ccccc3)cc2C(C)(C)c2cc(N(c3ccccc3)c3ccccc3)ccc21]

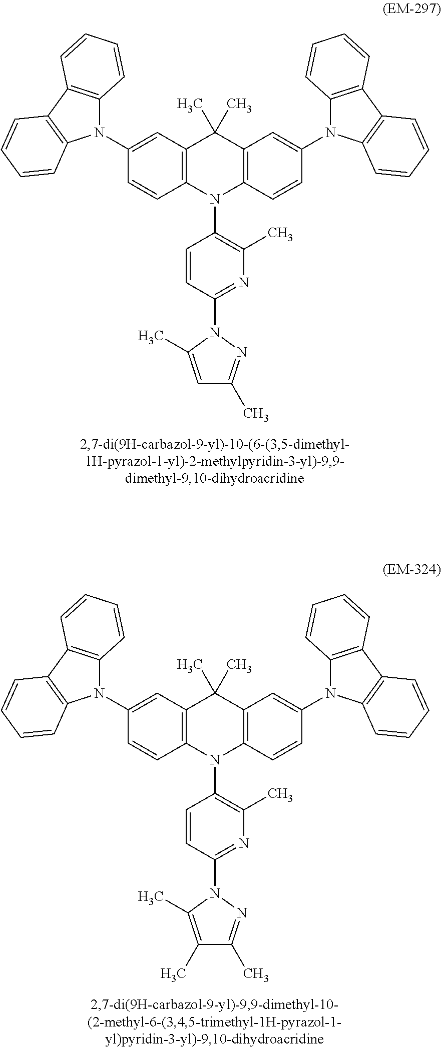 [SMILES: Cc1cc(C)n(-c2ccc(N3c4ccc(-n5c6ccccc6c6ccccc65)cc4C(C)(C)c4cc(-n5c6ccccc6c6ccccc65)ccc43)c(C)n2)n1 ; Cc1nc(-n2nc(C)c(C)c2C)ccc1N1c2ccc(-n3c4ccccc4c4ccccc43)cc2C(C)(C)c2cc(-n3c4ccccc4c4ccccc43)ccc21]

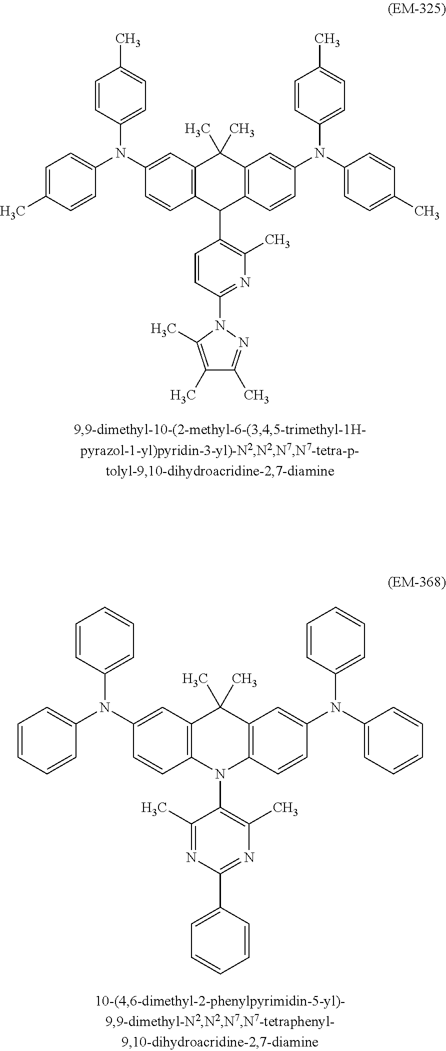

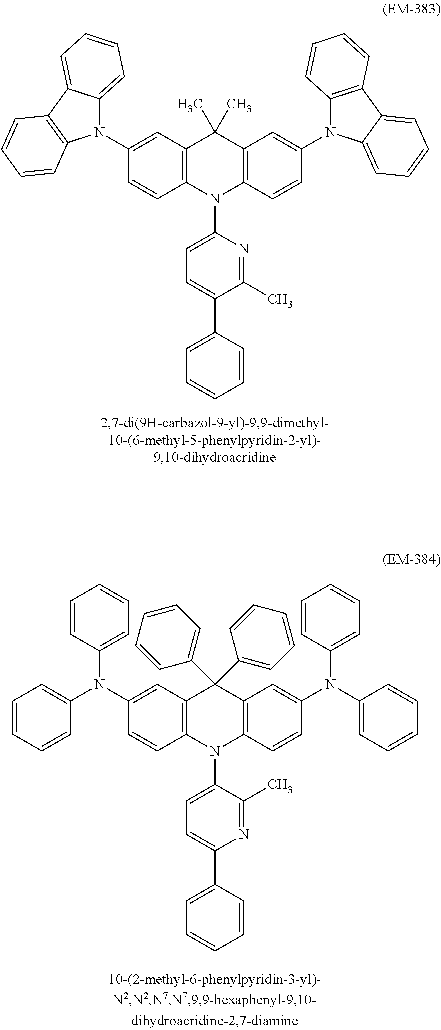 [SMILES: Cc1nc(N2c3ccc(-n4c5ccccc5c5ccccc54)cc3C(C)(C)c3cc(-n4c5ccccc5c5ccccc54)ccc32)ccc1-c1ccccc1 ; Cc1nc(-c2ccccc2)ccc1N1c2ccc(N(c3ccccc3)c3ccccc3)cc2C(c2ccccc2)(c2ccccc2)c2cc(N(c3ccccc3)c3ccccc3)ccc21]

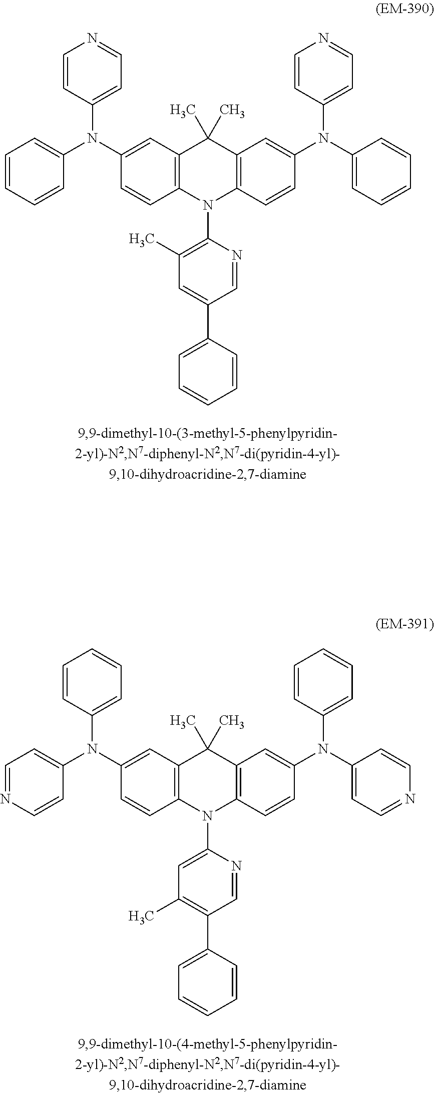 [SMILES: Cc1cc(-c2ccccc2)cnc1N1c2ccc(N(c3ccccc3)c3ccncc3)cc2C(C)(C)c2cc(N(c3ccccc3)c3ccncc3)ccc21 ; Cc1cc(N2c3ccc(N(c4ccccc4)c4ccncc4)cc3C(C)(C)c3cc(N(c4ccccc4)c4ccncc4)ccc32)ncc1-c1ccccc1]

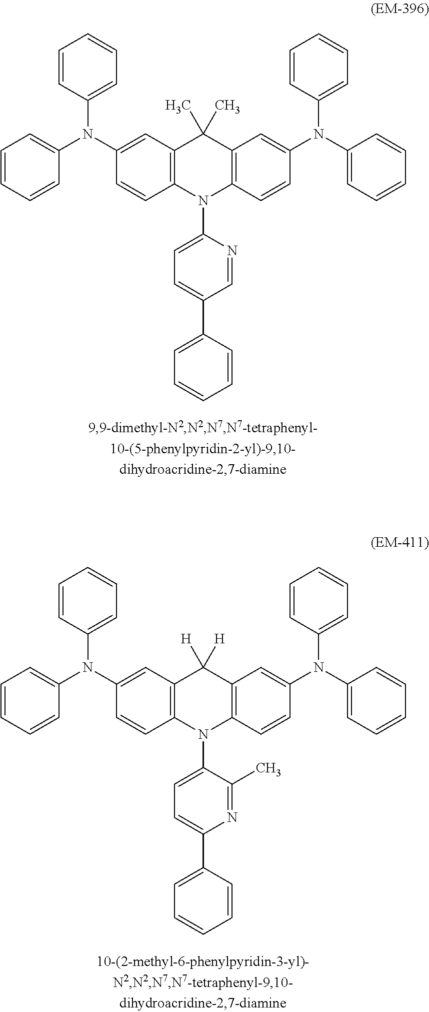 [SMILES: CC1(C)c2cc(N(c3ccccc3)c3ccccc3)ccc2N(c2ccc(-c3ccccc3)cn2)c2ccc(N(c3ccccc3)c3ccccc3)cc21 ; Cc1nc(-c2ccccc2)ccc1N1c2ccc(N(c3ccccc3)c3ccccc3)cc2Cc2cc(N(c3ccccc3)c3ccccc3)ccc21]

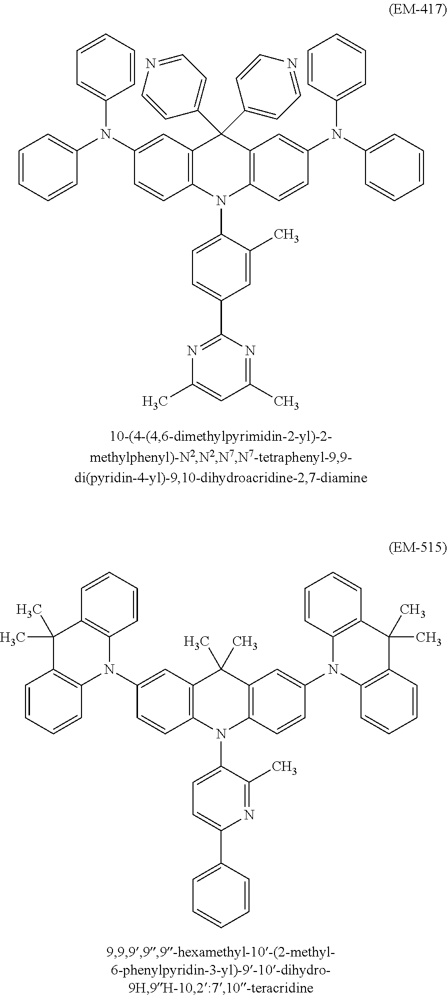

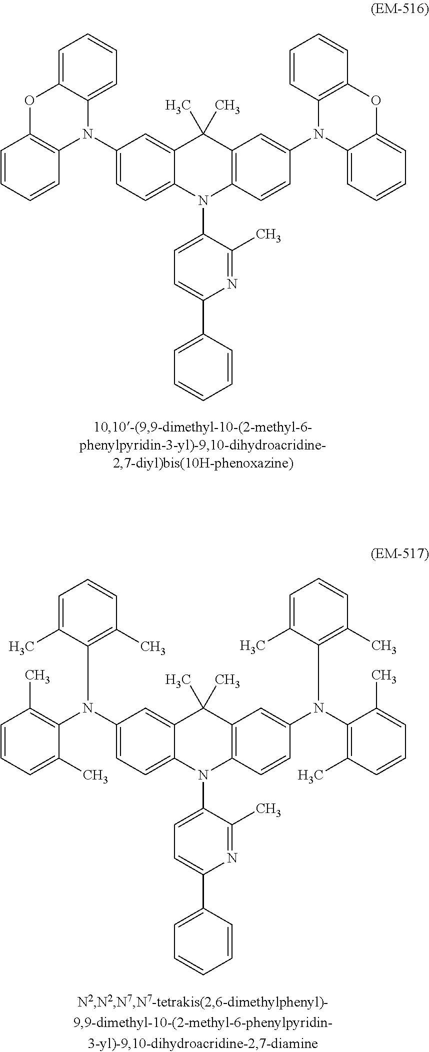 [SMILES: Cc1nc(-c2ccccc2)ccc1N1c2ccc(N3c4ccccc4Oc4ccccc43)cc2C(C)(C)c2cc(N3c4ccccc4Oc4ccccc43)ccc21 ; Cc1cccc(C)c1N(c1ccc2c(c1)C(C)(C)c1cc(N(c3c(C)cccc3C)c3c(C)cccc3C)ccc1N2c1ccc(-c2ccccc2)nc1C)c1c(C)cccc1C]

The compounds of this invention may be prepared by methods known in the art, e.g., by those methods illustrated in the examples and variations thereof that will be known to those skilled in the art.

Preferably, at least one compound of this invention is part of an optoelectronic device, e.g., an electroluminescent device, preferably in the emitter layer thereof. Preferably, at least one compound of this invention is used as a thermally activated delayed fluorescent (TADF) dopant, preferably in an OLED device. In some instances, the compound is attached to a polymer which forms a film which can be present in one, some, or all of the following layers: hole injection layer (HIL), a hole transport layer (HTL), an emitting material layer (EML), an electron transport layer (ETL), and an electron injection layer (EIL). Preferably, the film has a layer thickness of at least 5 nm, preferably at least 10 nm, preferably at least 20 nm, preferably no more than 90 nm, preferably no more than 80 nm, preferably no more than 70 nm, preferably no more than 60 nm, preferably no more than 50 nm. In an embodiment, the film is formed with an evaporative process. In an embodiment, the film is formed in a solution process.

Preferably, the electronic device is an OLED device and the present composition is a dopant in the emitting layer. When the present composition is the dopant, the host material has a triplet energy level higher than that of the doped emitter molecule. Suitable host materials can be found in Yook et al. “Organic Materials for Deep Blue Phosphorescent Organic Light-Emitting Diodes” Adv. Mater. 2012, 24, 3169-3190, and in Mi et al. “Molecular Hosts for Triplet Emitters in Organic Light-Emitting Diodes and the Corresponding Working Principle” Sci. China Chem. 2010, 53, 1679.

Preferably, compound(s) of the present invention are in the emitting layer of the OLED device and are present in a total amount of at least 1 wt %, preferably at least 5 wt %; preferably no more than 25 wt %, preferably no more than 30 wt %, preferably no more than 40.0 wt % based on the total weight of the emitting layer. Additional hosts or dopants can be present in the device or in the emitting layer.

Preferably, the OLED device contains compound(s) of the present invention in the emitting layer and the OLED device emits light by way of TADF. Preferably, the TADF-emitted light is visible light. Preferably, the energy difference between the first triplet state (T₁) and the singlet state (S₁) is less than 0.7 eV, preferably less than 0.6 eV, preferably less than 0.5 eV. More preferably, the energy difference is less than 0.30 eV. More preferably, the energy difference is less than 0.20 eV. Preferably, the calculated HOMO of the compound is higher than −5.5 eV, preferably higher than −5.3 eV, preferably higher than −5.2 eV, preferably higher than −5.1 eV, preferably higher than −5 eV, preferably higher than −4.9 eV.

### Examples

### Synthesis of Materials

### Synthesis of 9,9-Dimethyl-9,10-dihydroacridine

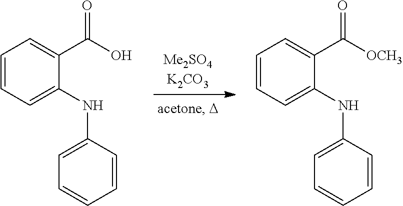 [SMILES: O=C(O)c1ccccc1Nc1ccccc1 ; COC(=O)c1ccccc1Nc1ccccc1]

### Methyl 2-(phenylamino)benzoate

To a flask charged with N-phenyl-anthranilic acid (10.0 g, 46.9 mmol) and potassium carbonate (6.48 g, 46.9 mmol) in acetone (140 mL) was added dimethyl sulfate (7.56 mL, 79.7 mmol) at room temperature. The flask was attached to a reflux condenser and heated to reflux. After 2 h, the was cooled to rt, and poured onto crushed ice. The resulting mixture was extracted with dichloromethane and then dried over Na₂SO₄. The resulting solution was filtered and concentrated. The resulting residue was purified via silica gel chromatography, eluted with hexane:ethyl acetate=6:4, to afford the product as a yellow oil in 94% yield.

¹H NMR (400 MHz, CDCl₃) δ 9.47 (s, 1H), 7.96 (dd, J=7.8, 1.5 Hz, 1H), 7.38-7.22 (m, 6H), 7.14-7.05 (m, 1H), 6.73 (ddd, J=8.1, 6.9, 1.4 Hz, 1H), 3.90 (s, 3H).

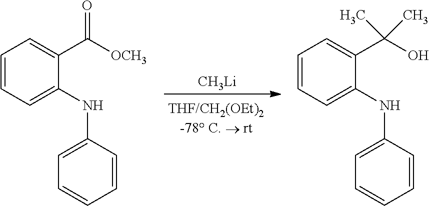 [SMILES: COC(=O)c1ccccc1Nc1ccccc1 ; "CH3Li" ; CC(C)(O)c1ccccc1Nc1ccccc1]

### 2-(2-(Phenylamino)phenyl)propan-2-ol

To a flask charged with methyl 2-(phenylamino)benzoate (5.00 g, 22.0 mmol) was added THF (50 mL). The flask was cooled to −78° C. and a methyl lithium solution (3.0 M in diethoxymethane, 22.0 mL, 66.0 mmol) was added over 30 min. The reaction was stirred at −78° C. After 30 min, the flask was removed from the −78° C. bath and stirred at rt. After 1 h, the reaction mixture was quenched with ice water, and then extracted with ethyl acetate. The organic layer was washed with water and brine, and then dried over MgSO₄. The resulting solution was filtered and concentrated to afford the product as in 99% yield.

¹H NMR (400 MHz, DMSO-d₆) δ 8.47 (s, 1H), 7.30-7.19 (m, 4H), 7.15 (ddd, J=8.2, 7.3, 1.5 Hz, 1H), 6.98 (dd, J=8.6, 1.2 Hz, 2H), 6.89-6.77 (m, 2H), 5.82 (s, 1H), 1.51 (s, 6H).

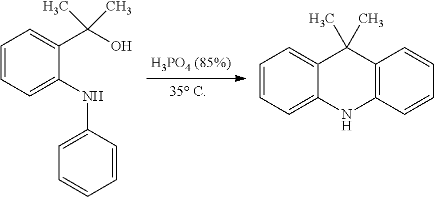 [SMILES: CC(C)(O)c1ccccc1Nc1ccccc1 ; CC1(C)c2ccccc2Nc2ccccc21]

### 9,9-Dimethyl-9,10-dihydroacridine

To a flask charged with 2-(2-(phenylamino)phenyl)propan-2-ol (5.00 g, 22.0 mmol) was added phosphoric acid (85%, 76 mL). The reaction was stirred and heated to 35° C. After 2 h, the crude reaction mixture was cooled to room temperature and slowly poured onto ice. The mixture was extracted with dichloromethane. The combined organic layers were washed with water and brine. The washed organic layer was dried over Na₂SO₄, filtered and concentrated. The resulting solid product, afforded in 87% yield, was used without further purification.

¹H NMR (400 MHz, CDCl₃) δ 7.28 (dd, J=7.8, 1.4 Hz, 2H), 7.10 (ddd, J=7.9, 7.2, 1.4 Hz, 2H), 6.92 (ddd, J=7.9, 7.0, 1.3 Hz, 2H), 6.70 (dd, J=7.8, 1.2 Hz, 2H), 6.14 (s, 1H), 1.58 (s, 6H).

### Synthesis of EM-06

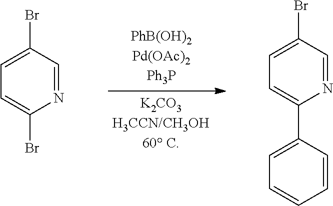 [SMILES: Brc1ccc(Br)nc1 ; Brc1ccc(-c2ccccc2)nc1]

### 5-Bromo-2-phenylpyridine

To a flask charged with 2,5-dibromopyridine (5.00 g, 21.1 mmol), phenyl boronic acid (2.83 g, 23.2 mmol), palladium acetate (237 mg, 1.06 mmol), triphenylphosphine (554 mg, 2.11 mmol), and potassium carbonate (5.83 g, 42.2 mmol) under a N₂ atmosphere was added acetonitrile (150 mL) and methanol (75 mL) at room temperature. The flask was attached to a reflux condenser and heated to 60° C. After 24 h, the crude reaction mixture was cooled to rt, and the volatiles were removed under reduced pressure. The resulting residue was dissolved in dichloromethane, and the organic layer was washed with water and brine. The washed organic layer was dried over Na₂SO₄, filtered and concentrated. The resulting residue was purified via silica gel chromatography, eluted with hexane:CH₂Cl₂=5:1, to afford the product as a white solid in 81% yield.

¹H NMR (400 MHz, CDCl₃) δ 8.74 (dd, J=2.5, 0.8 Hz, 1H), 7.96 (dd, J=8.2, 1.5 Hz, 2H), 7.87 (dd, J=8.5, 2.4 Hz, 1H), 7.63 (dd, J=8.5, 0.8 Hz, 1H), 7.52-7.39 (m, 3H).

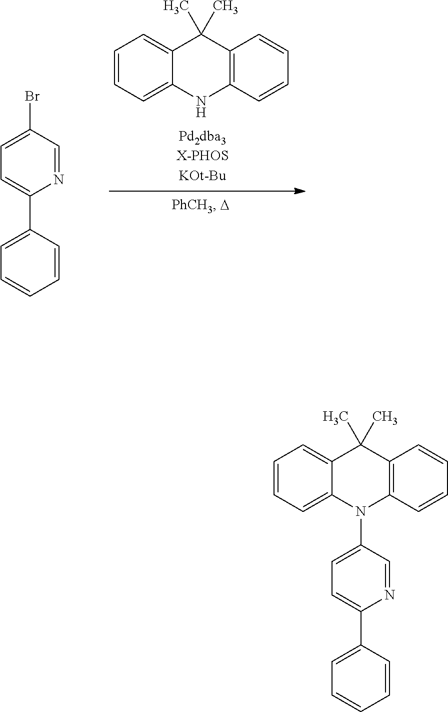 [SMILES: Brc1ccc(-c2ccccc2)nc1 ; CC1(C)c2ccccc2Nc2ccccc21 ; CC1(C)c2ccccc2N(c2ccc(-c3ccccc3)nc2)c2ccccc21]

### 9,9-Dimethyl-10-(6-phenylpyridin-3-yl)-9,10-dihydroacridine

To a flask charged with 5-bromo-2-phenylpyridine (2.00 g, 9.56 mmol), 9,9-dimethyl-9,10-dihydroacridine (2.69 g, 11.5 mmol), tris(dibenzylideneacetone) dipalladium (175 mg, 0.191 mmol), 2-(dicyclohexylphosphino)-2′,4′,6′-tri-i-propyl-1,1′-biphenyl (X-PHOS) (273 mg, 0.573 mmol), and potassium t-butoxide (2.15 g, 19.1 mmol) under a N₂ atmosphere was added toluene (75 mL) at room temperature. The flask was attached to a reflux condenser and heated to reflux. After 24 h, the crude reaction mixture was cooled to rt, diluted with water, and extracted with ethyl acetate. The organic layer was washed with water and brine, and then dried over MgSO₄. The resulting solution was filtered and concentrated. The resulting residue was purified via silica gel chromatography, eluted with hexane:CH₂Cl₂=1:1, to afford the product as a white solid in 71% yield.

¹H NMR (400 MHz, CDCl₃) δ 8.68 (dd, J=2.5, 0.8 Hz, 1H), 8.15-8.08 (m, 2H), 8.02 (dd, J=8.3, 0.8 Hz, 1H), 7.78 (dd, J=8.4, 2.5 Hz, 1H), 7.58-7.453 (m, 5H), 7.05-6.93 (m, 4H), 6.32 (dd, J=7.6, 1.7 Hz, 2H), 1.72 (s, 6H).

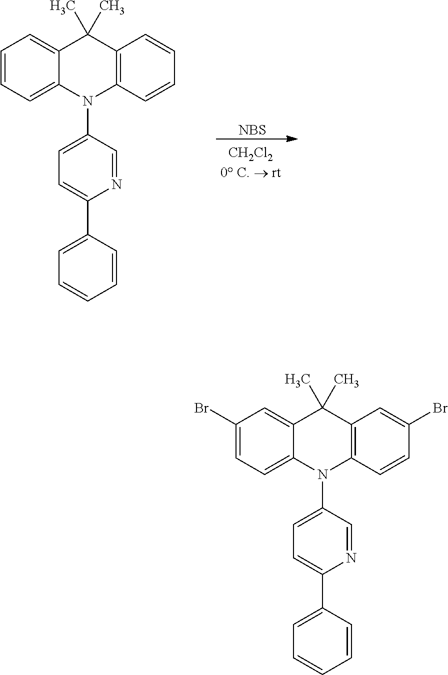 [SMILES: CC1(C)c2ccccc2N(c2ccc(-c3ccccc3)nc2)c2ccccc21 ; "NBS" ; CC1(C)c2cc(Br)ccc2N(c2ccc(-c3ccccc3)nc2)c2ccc(Br)cc21]

### 2,7-Dibromo-9,9-dimethyl-10-(6-phenylpyridin-3-yl)-9,10-dihydroacridine

To a flask charged with 9,9-dimethyl-10-(6-phenylpyridin-3-yl)-9,10-dihydroacridine (2.45 g, 6.76 mmol) was added dichoromethane (100 mL) at room temperature. The flask was cooled to 0° C. and N-bromosuccinimide (2.53 g, 14.2 mmol) was added in 5 portions over 10 min. The reaction was stirred at 0° C. for 1 h, and then stirred at rt for 2 h. The crude reaction mixture was diluted with water, and extracted with CH₂Cl₂. The combined organic layers were washed with water and brine, and then dried over Na₂SO₄. The resulting solution was filtered and concentrated. The resulting residue was purified via silica gel chromatography, eluted with hexane:CH₂Cl₂=1:1, to afford the product as a white solid in 85% yield.

¹H NMR (400 MHz, CDCl₃) δ8.63 (dd, J=2.6, 0.8 Hz, 1H), 8.17-8.08 (m, 2H), 8.03 (dd, J=8.4, 0.8 Hz, 1H), 7.73 (dd, J=8.4, 2.5 Hz, 1H), 7.60-7.44 (m, 5H), 7.10 (dd, J=8.8, 2.3 Hz, 2H), 6.19 (d, J=8.8 Hz, 2H), 1.67 (s, 6H).

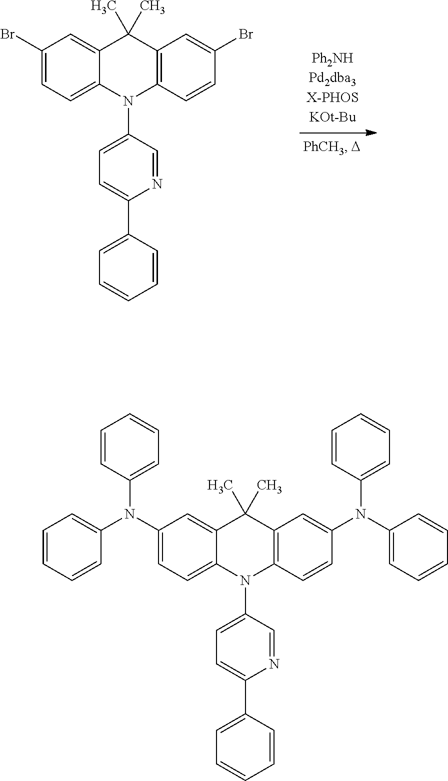 [SMILES: CC1(C)c2cc(Br)ccc2N(c2ccc(-c3ccccc3)nc2)c2ccc(Br)cc21 ; CC1(C)c2cc(N(c3ccccc3)c3ccccc3)ccc2N(c2ccc(-c3ccccc3)nc2)c2ccc(N(c3ccccc3)c3ccccc3)cc21]

### 9,9-Dimethyl-N²,N²,N⁷,N⁷-tetraphenyl-10-(6-phenylpyridin-3-yl)-9,10-dihydroacridine-2,7-diamine (EM-06)

To a flask charged with 2,7-dibromo-9,9-dimethyl-10-(6-phenylpyridin-3-yl)-9,10-dihydroacridine (3.00 g, 5.74 mmol), diphenylamine (2.33 g, 13.8 mmol), tris(dibenzylideneacetone) dipalladium (210 mg, 0.230 mmol), 2-(dicyclohexylphosphino)-2′,4′,6′-tri-i-propyl-1,1′-biphenyl (X-PHOS) (329 mg, 0.689 mmol), and potassium t-butoxide (2.58 g, 23.0 mmol) under a N₂ atmosphere was added toluene (70 mL) at room temperature. The flask was attached to a reflux condenser and heated to reflux. After 24 h, the crude reaction mixture was cooled to rt, diluted with water, and extracted with ethyl acetate. The organic layer was washed with water and brine, and then dried over MgSO₄. The resulting solution was filtered and concentrated. The resulting residue was purified via silica gel chromatography, eluted with hexane:CH₂Cl₂=1:2, to afford the product as a pale yellow solid in 80% yield. The material was recrystallized from hot methanol to afford a yellow solid.

¹H NMR (400 MHz, C₆D₆) δ 8.68 (d, J=2.4 Hz, 1H), 8.25-8.17 (m, 2H), 7.46-7.38 (m, 3H), 7.32-7.26 (m, 2H), 7.20 (dd, J=8.4, 2.4 Hz, 2H), 7.18-7.12 (m, 8H), 7.09-7.03 (m, 8H), 6.81 (tt, J=7.4, 1.4 Hz, 4H), 6.74 (dd, J=8.8, 2.4 Hz, 2H), 6.19 (d, J=8.8 Hz, 2H), 1.36 (s, 6H).

### Synthesis of EM-64

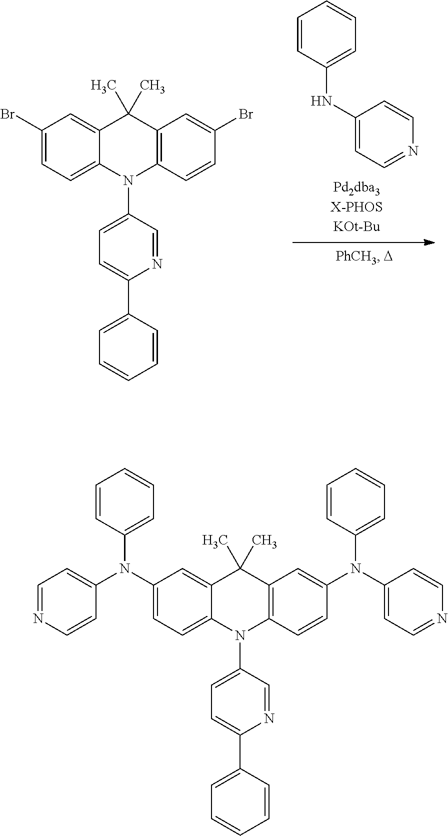 [SMILES: CC1(C)c2cc(Br)ccc2N(c2ccc(-c3ccccc3)nc2)c2ccc(Br)cc21 ; c1ccc(Nc2ccncc2)cc1 ; CC1(C)c2cc(N(c3ccccc3)c3ccncc3)ccc2N(c2ccc(-c3ccccc3)nc2)c2ccc(N(c3ccccc3)c3ccncc3)cc21]

### 9,9-Dimethyl-N²,N⁷-diphenyl-10-(6-phenylpyridin-3-yl)-N²,N⁷-di(pyridin-4-yl)-9,10-dihydroacridine-2,7-diamine (EM-64)

To a flask charged with 2,7-dibromo-9,9-dimethyl-10-(6-phenylpyridin-3-yl)-9,10-dihydroacridine (1.04 g, 2.00 mmol), N-phenylpyridin-4-amine (817 mg, 4.80 mmol), tris(dibenzylideneacetone) dipalladium (73 mg, 0.08 mmol), 2-(dicyclohexylphosphino)-2′,4′,6′-tri-i-propyl-1,1′-biphenyl (X-PHOS) (114 mg, 0.24 mmol), and potassium t-butoxide (897 mg, 8.0 mmol) under a N₂ atmosphere was added toluene (30 mL) at room temperature. The flask was attached to a reflux condenser and heated to reflux. After 24 h, the crude reaction mixture was cooled to rt, diluted with water, and extracted with ethyl acetate. The organic layer was washed with water and brine, and then dried over MgSO₄. The resulting solution was filtered and concentrated. The resulting residue was purified via silica gel chromatography, eluted with CH₂Cl₂, then EtOAc, then EtOAc:Et₃N=98:2, to afford the product as a yellow solid in 57% yield. The material was recrystallized from CH₂Cl₂/hexane to afford an off-white solid.

¹H NMR (400 MHz, CDCl₃) δ 8.71 (d, J=2.4 Hz, 1H), 8.21 (dd, J=4.8, 1.6 Hz, 4H), 8.09 (br d, J=6.8 Hz, 2H), 8.02 (d, J=8.4 Hz, 1H), 7.81 (dd, J=8.4, 2.5 Hz, 1H), 7.59-7.44 (m, 3H), 7.38-7.29 (m, 5H), 7.22-7.13 (m, 5H), 6.82 (dd, J=8.8, 2.4 Hz, 2H), 6.70 (dd, J=5.2, 2.0 Hz, 4H), 6.32 (d, J=8.7 Hz, 2H), 1.58 (s, 6H).

### Synthesis of EM-07

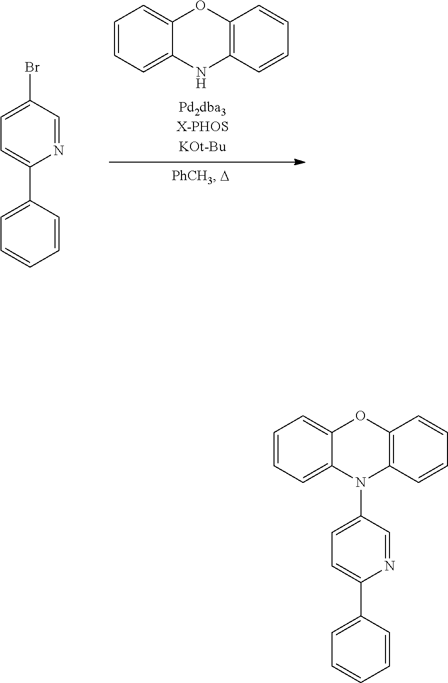 [SMILES: Brc1ccc(-c2ccccc2)nc1 ; c1ccc2c(c1)Nc1ccccc1O2 ; c1ccc(-c2ccc(N3c4ccccc4Oc4ccccc43)cn2)cc1]

### 10-(6-Phenylpyridin-3-yl)-10H-phenoxazine

To a flask charged with 5-bromo-2-phenylpyridine (2.81 g, 12.0 mmol), phenoxazine (2.00 g, 10.9 mmol), tris(dibenzylideneacetone) dipalladium (200 mg, 0.218 mmol), 2-(dicyclohexylphosphino)-2′,4′,6′-tri-i-propyl-1,1′-biphenyl (X-PHOS) (312 mg, 0.655 mmol), and potassium t-butoxide (2.45 g, 21.8 mmol) under a N₂ atmosphere was added toluene (80 mL) at room temperature. The flask was attached to a reflux condenser and heated to reflux. After 24 h, the crude reaction mixture was cooled to rt, diluted with water, and extracted with ethyl acetate. The organic layer was washed with water and brine, and then dried over MgSO₄. The resulting solution was filtered and concentrated. The resulting residue was purified via silica gel chromatography, eluted with hexane:CH₂Cl₂=1:1, to afford the product as a white solid in 83% yield.

¹H NMR (400 MHz, CDCl₃) δ 8.69 (dd, J=2.5, 0.8 Hz, 1H), 8.13-8.03 (m, 2H), 7.98 (dd, J=8.4, 0.8 Hz, 1H), 7.79 (dd, J=8.4, 2.5 Hz, 1H), 7.58-7.42 (m, 3H), 6.76-6.60 (m, 6H), 6.00 (dd, J=7.9, 1.5 Hz, 2H).

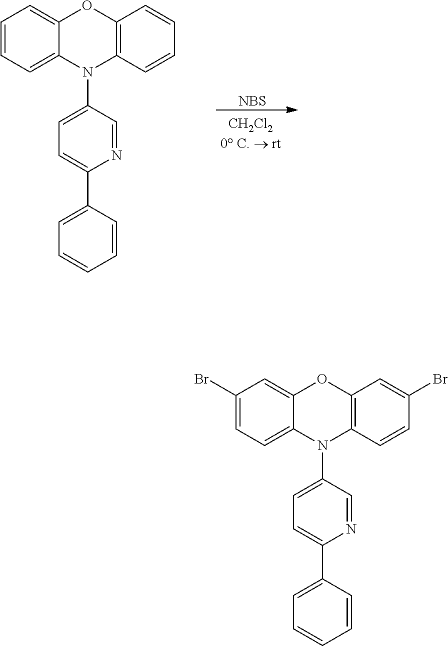 [SMILES: c1ccc(-c2ccc(N3c4ccccc4Oc4ccccc43)cn2)cc1 ; "NBS" ; Brc1ccc2c(c1)Oc1cc(Br)ccc1N2c1ccc(-c2ccccc2)nc1]

### 3,7-Dibromo-10-(6-phenylpyridin-3-yl)-10H-phenoxazine

To a flask charged with 10-(6-phenylpyridin-3-yl)-10H-phenoxazine (2.37 g, 7.05 mmol) was added dichoromethane (100 mL) at room temperature. The flask was cooled to 0° C. and N-bromosuccinimide (2.76 g, 15.5 mmol) was added in 5 portions over 10 min. The reaction was stirred at 0° C. for 1 h, and then stirred at rt for 2 h. The crude reaction mixture was diluted with water, and extracted with CH₂Cl₂. The combined organic layers were washed with water and brine, and then dried over Na₂SO₄. The resulting solution was filtered and concentrated. The resulting residue was purified via silica gel chromatography, eluted with hexane:CH₂Cl₂=1:1, to afford the product as a white solid in 78% yield.

¹H NMR (400 MHz, CDCl₃) δ8.64 (dd, J=2.5, 0.8 Hz, 1H), 8.12-8.02 (m, 2H), 7.99 (dd, J=8.4, 0.8 Hz, 1H), 7.74 (dd, J=8.4, 2.5 Hz, 1H), 7.58-7.43 (m, 3H), 6.87 (d, J=2.2 Hz, 2H), 6.75 (dd, J=8.6, 2.2 Hz, 2H), 5.85 (d, J=8.6 Hz, 2H).

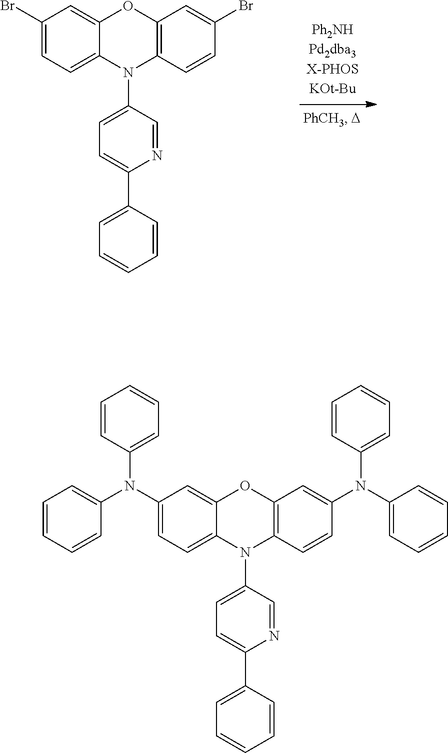 [SMILES: Brc1ccc2c(c1)Oc1cc(Br)ccc1N2c1ccc(-c2ccccc2)nc1 ; c1ccc(-c2ccc(N3c4ccc(N(c5ccccc5)c5ccccc5)cc4Oc4cc(N(c5ccccc5)c5ccccc5)ccc43)cn2)cc1]

### N³,N³,N⁷,N⁷-Tetraphenyl-10-(6-phenylpyridin-3-yl)-10H-phenoxazine-3,7-diamine (EM 07)

To a flask charged with 3,7-dibromo-10-(6-phenylpyridin-3-yl)-4a, 10a-dihydro-10H-phenoxazine (2.73 g, 5.50 mmol), diphenylamine (2.23 g, 13.2 mmol), tris(dibenzylideneacetone) dipalladium (201 mg, 0.220 mmol), 2-(dicyclohexylphosphino)-2′,4′,6′-tri-i-propyl-1,1′-biphenyl (X-PHOS) (315 mg, 0.66 mmol), and potassium t-butoxide (2.47 g, 22.0 mmol) under a N₂ atmosphere was added toluene (70 mL) at room temperature. The flask was attached to a reflux condenser and heated to reflux. After 24 h, the crude reaction mixture was cooled to rt, diluted with water, and extracted with ethyl acetate. The organic layer was washed with water and brine, and then dried over MgSO₄. The resulting solution was filtered and concentrated. The resulting residue was purified via silica gel chromatography, eluted with hexane:CH₂Cl₂=1:2, to afford the product as an off-white solid in 81% yield. The material was recrystallized from hot methanol to afford a white solid.

¹H NMR (400 MHz, CDCl₃) δ 8.71 (d, J=1.6 Hz, 1H), 8.10-8.01 (m, 2H), 7.74 (d, J=8.4 Hz, 1H), 7.23 (dd, J=8.4, 2.4 Hz, 1H), 7.57-7.41 (m, 3H), 7.27-7.14 (m, 8H), 7.11-7.00 (m, 8H), 6.95 (t, J=7.3 Hz, 4H), 6.49 (d, J=2.5 Hz, 2H), 6.39 (d, J=6.0 Hz, 1H), 5.93 (d, J=8.6 Hz, 2H).

### Synthesis of EM-01

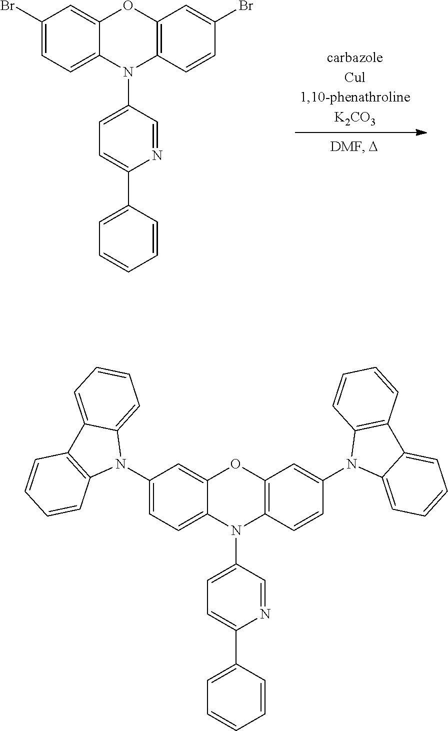 [SMILES: Brc1ccc2c(c1)Oc1cc(Br)ccc1N2c1ccc(-c2ccccc2)nc1 ; c1ccc(-c2ccc(N3c4ccc(-n5c6ccccc6c6ccccc65)cc4Oc4cc(-n5c6ccccc6c6ccccc65)ccc43)cn2)cc1]

### 3,7-Di(9H-carbazol-9-yl)-10-(6-phenylpyridin-3-yl)-10H-phenoxazine (EM-01)

To a flask charged with 3,7-dibromo-10-(6-phenylpyridin-3-yl)-4a, 10a-dihydro-10H-phenoxazine (1.82 g, 3.67 mmol), carbazole (1.41 g, 8.44 mmol), copper iodide (140 mg, 0.735 mmol), 1,10-phenathroline (265 mg, 1.47 mmol), and potassium carbonate (2.28 g, 16.5 mmol) under a N₂ atmosphere was added dimethylformamide (20 mL) at room temperature. The flask was attached to a reflux condenser and heated to reflux. After 24 h, the crude reaction mixture was cooled to rt, diluted with water, and extracted with chloroform. The organic layer was washed with water and brine, and then dried over Na₂SO₄. The resulting solution was filtered and concentrated. The resulting residue was purified via silica gel chromatography, eluted with hexane:CH₂Cl₂=1:2, to afford the product as an off-white solid in 69% yield. The material was recrystallized from hot methanol to afford a white solid.

¹H NMR (400 MHz, CDCl₃) δ 8.89 (d, J=1.6 Hz, 1H), 8.17-8.02 (m, 7H), 7.99 (dd, J=8.4, 2.5 Hz, 1H), 7.61-7.47 (m, 3H), 7.47-7.38 (m, 8H), 7.32-7.22 (m, 4H), 6.99 (d, J=2.3 Hz, 2H), 6.89 (dd, J=8.5, 2.3 Hz, 2H), 6.26 (d, J=8.5 Hz, 2H).

### Synthesis of EM-08

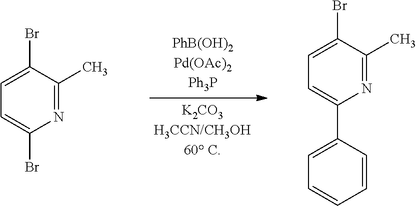

### 3-Bromo-2-methyl-6-phenylpyridine

To a flask charged with 3,6-dibromo-2-methylpyridine (4.69 g, 18.7 mmol), phenyl boronic acid (2.62 g, 21.5 mmol), palladium acetate (210 mg, 0.935 mmol), triphenylphosphine (490 mg, 1.87 mmol), and potassium carbonate (5.17 g, 37.4 mmol) under a N₂ atmosphere was added acetonitrile (130 mL) and methanol (65 mL) at room temperature. The flask was attached to a reflux condenser and heated to 60° C. After 24 h, the crude reaction mixture was cooled to rt, and the volatiles were removed under reduced pressure. The resulting residue was dissolved in dichloromethane, and the organic layer was washed with water and brine. The washed organic layer was dried over Na₂SO₄, filtered and concentrated. The resulting residue was purified via silica gel chromatography, eluted with hexane:CH₂Cl₂=5:1, to afford the product as a white solid in 64% yield.

¹H NMR (400 MHz, CDCl₃) δ 8.00-7.93 (m, 2H), 7.84 (d, J=8.2 Hz, 1H), 7.51-7.38 (m, 4H), 2.74 (s, 3H).

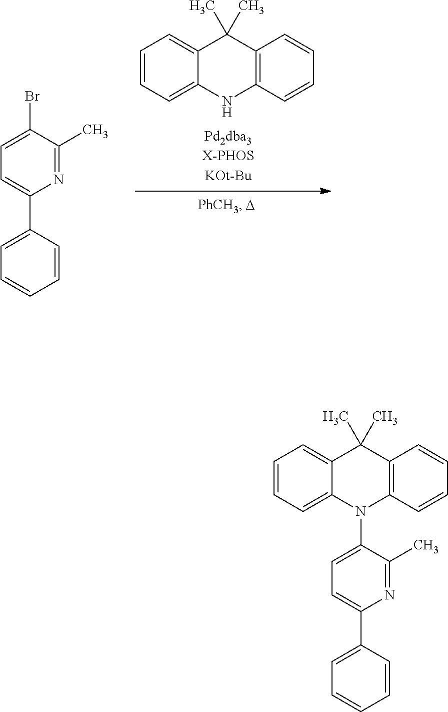 [SMILES: Cc1nc(-c2ccccc2)ccc1Br ; CC1(C)c2ccccc2Nc2ccccc21 ; Cc1nc(-c2ccccc2)ccc1N1c2ccccc2C(C)(C)c2ccccc21]

### 9,9-Dimethyl-10-(2-methyl-6-phenylpyridin-3-yl)-9,10-dihydroacridine

To a flask charged with 3-bromo-2-methyl-6-phenylpyridine (2.13 g, 8.60 mmol), 9,9-dimethyl-9,10-dihydroacridine (1.50 g, 7.17 mmol), tris(dibenzylideneacetone) dipalladium (131 mg, 0.143 mmol), 2-(dicyclohexylphosphino)-2′,4′,6′-tri-i-propyl-1,1′-biphenyl (X-PHOS) (205 mg, 0.430 mmol), and potassium t-butoxide (1.61 g, 14.3 mmol) under a N₂ atmosphere was added toluene (60 mL) at room temperature. The flask was attached to a reflux condenser and heated to reflux. After 24 h, the crude reaction mixture was cooled to rt, diluted with water, and extracted with ethyl acetate. The organic layer was washed with water and brine, and then dried over MgSO₄. The resulting solution was filtered and concentrated. The resulting residue was purified via silica gel chromatography, eluted with hexane:CH₂Cl₂=1:1, to afford the product as a white solid in 82% yield.

¹H NMR (400 MHz, CDCl₃) δ 8.14-8.10 (m, 2H), 7.84 (d, J=8.1 Hz, 1H), 7.65 (d, J=8.1 Hz, 1H), 7.56-7.43 (m, 5H), 7.03-6.93 (m, 4H), 6.18 (dd, J=8.1, 1.4 Hz, 2H), 2.41 (s, 3H), 1.76 (s, 3H), 1.69 (s, 3H).

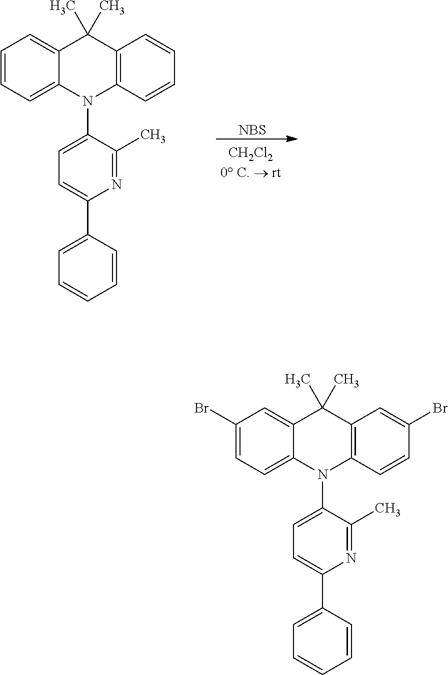 [SMILES: Cc1nc(-c2ccccc2)ccc1N1c2ccccc2C(C)(C)c2ccccc21 ; "NBS" ; Cc1nc(-c2ccccc2)ccc1N1c2ccc(Br)cc2C(C)(C)c2cc(Br)ccc21]

### 2,7-Dibromo-9,9-dimethyl-10-(2-methyl-6-phenylpyridin-3-yl)-9,10-dihydroacridine

To a flask charged with 9,9-dimethyl-10-(2-methyl-6-phenylpyridin-3-yl)-9, 10-dihydroacridine (2.16 g, 5.72 mmol) was added dichoromethane (85 mL) at room temperature. The flask was cooled to 0° C. and N-bromosuccinimide (2.14 g, 12.0 mmol) was added in 5 portions over 10 min. The reaction was stirred at 0° C. for 1 h, and then stirred at rt for 2 h. The crude reaction mixture was diluted with water, and extracted with CH₂Cl₂. The combined organic layers were washed with water and brine, and then dried over Na₂SO₄. The resulting solution was filtered and concentrated. The resulting residue was purified via silica gel chromatography, eluted with hexane:CH₂Cl₂=1:1, to afford the product as a white solid in 90% yield.

¹H NMR (400 MHz, CDCl₃) δ 8.15-8.08 (m, 2H), 7.84 (d, J=8.2 Hz, 1H), 7.60 (d, J=8.2 Hz, 1H), 7.57-7.44 (m, 5H), 7.09 (dd, J=8.8, 2.3 Hz, 2H), 6.06 (d, J=8.1 Hz, 2H), 2.36 (s, 3H), 1.70 (s, 3H), 1.66 (s, 3H).

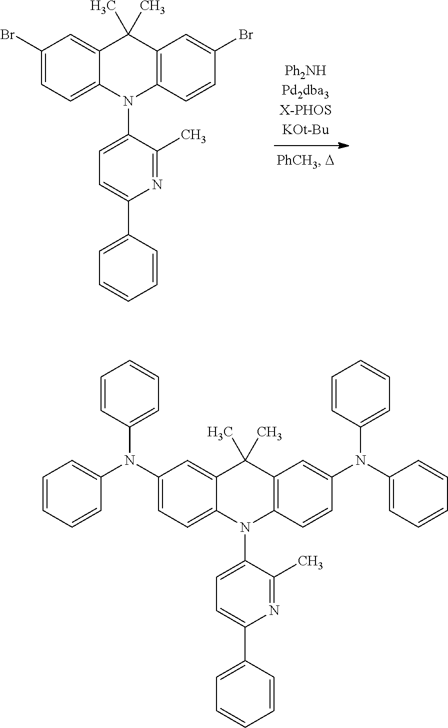 [SMILES: Cc1nc(-c2ccccc2)ccc1N1c2ccc(Br)cc2C(C)(C)c2cc(Br)ccc21 ; Cc1nc(-c2ccccc2)ccc1N1c2ccc(N(c3ccccc3)c3ccccc3)cc2C(C)(C)c2cc(N(c3ccccc3)c3ccccc3)ccc21]

### 9,9-Dimethyl-10-(2-methyl-6-phenylpyridin-3-yl)-N²,N²,N⁷,N⁷-tetraphenyl-9,10-dihydroacridine-2,7-diamine (EM-08)

To a flask charged with 2,7-dibromo-9,9-dimethyl-10-(2-methyl-6-phenylpyridin-3-yl)-9,10-dihydroacridine (2.74 g, 5.11 mmol), diphenylamine (2.08 g, 12.3 mmol), tris(dibenzylideneacetone) dipalladium (187 mg, 0.204 mmol), 2-(dicyclohexylphosphino)-2′,4′,6′-tri-i-propyl-1,1′-biphenyl (X-PHOS) (292 mg, 0.613 mmol), and potassium t-butoxide (2.29 g, 20.4 mmol) under a N₂ atmosphere was added toluene (65 mL) at room temperature. The flask was attached to a reflux condenser and heated to reflux. After 24 h, the crude reaction mixture was cooled to rt, diluted with water, and extracted with ethyl acetate. The organic layer was washed with water and brine, and then dried over MgSO₄. The resulting solution was filtered and concentrated. The resulting residue was purified via silica gel chromatography, eluted with hexane:CH₂Cl₂=1:2, to afford the product as a pale yellow solid in 88% yield. The material was recrystallized from hot methanol to afford an off-white solid, which was found to have a photoluminescent quantum efficiency of 61% in a PMMA film.

¹H NMR (400 MHz, acetone-d₆) δ 8.29-8.22 (m, 2H), 8.11 (d, J=8.2 Hz, 1H), 7.86 (d, J=8.2 Hz, 1H), 7.58-7.45 (m, 3H), 7.34 (d, J=2.5 Hz, 2H), 7.29-7.23 (m, 2H), 7.29-7.22 (m, 8H), 7.05-7.00 (m, 8H), 6.96 (tt, J=7.3, 1.2 Hz, 4H), 6.81 (dd, J=8.8, 2.5 Hz, 2H), 6.23 (d, J=8.8 Hz, 2H), 2.41 (s, 3H), 1.56 (s, 3H), 1.55 (s, 3H).

### Synthesis of EM-23

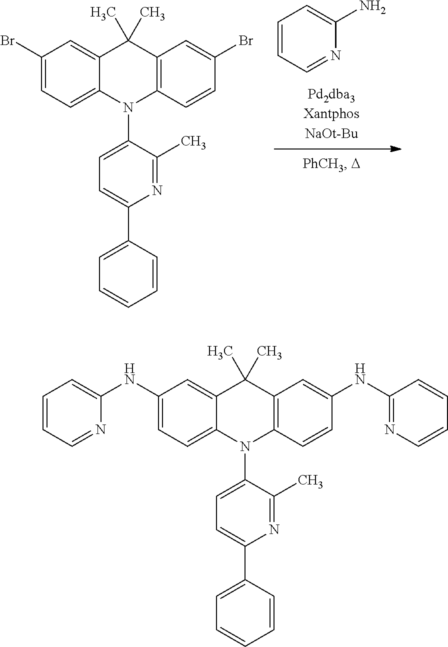

### 9,9-Dimethyl-10-(2-methyl-6-phenylpyridin-3-yl)-N²,N⁷-di(pyridin-2-yl)-9,10-dihydroacridine-2,7-diamine

To a flask charged with 2,7-dibromo-9,9-dimethyl-10-(2-methyl-6-phenylpyridin-3-yl)-9,10-dihydroacridine (1.36 g, 2.55 mmol), 2-amino pyridine (5.75 g, 6.11 mmol), tris(dibenzylideneacetone) dipalladium (47 mg, 0.051 mmol), 4,5-bis(diphenylphosphino)-9,9-dimethylxanthene (XantPhos) (33 mg, 0.056 mmol), and sodium t-butoxide (978 mg, 10.2 mmol) under a N₂ atmosphere was added toluene (40 mL) at room temperature. The flask was attached to a reflux condenser and heated to reflux. After 18 h, the crude reaction mixture was cooled to rt, diluted with water, and extracted with ethyl acetate. The organic layer was washed with water and brine, and then dried over MgSO₄. The resulting solution was filtered and concentrated. The resulting residue was purified via silica gel chromatography, eluted with hexane:CH₂Cl₂=1:5, to afford the product as a off-white solid in 42% yield.

¹H NMR (400 MHz, CDCl₃) δ 8.19-8.14 (m, 2H), 8.14-8.09 (m, 2H), 7.85 (d, J=8.2 Hz, 1H), 7.67 (d, J=8.2 Hz, 1H), 7.57-7.50 (m, 2H), 7.50-7.37 (m, 5H), 6.98 (d, J=7.6 Hz, 2H), 6.83-6.58 (m, 4H), 6.50 (s, 2H), 6.18 (d, J=8.8 Hz, 2H), 2.45 (s, 3H), 1.75 (s, 3H), 1.71 (s, 3H).

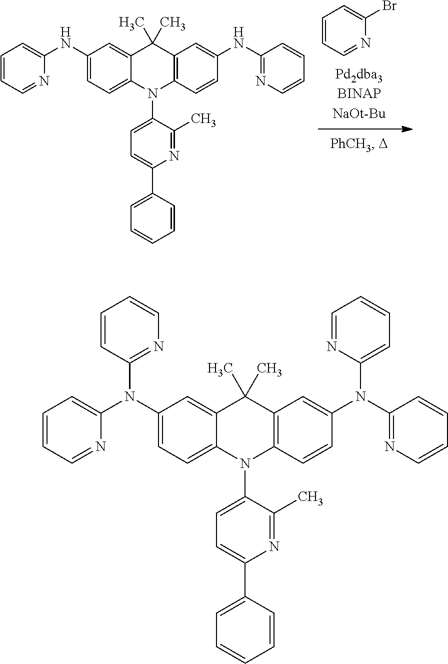

### 9,9-Dimethyl-10-(2-methyl-6-phenylpyridin-3-yl)-N²,N²,N⁷,N⁷-tetra(pyridin-2-yl)-9, 10-dihydroacridine-2,7-diamine

To a flask charged with 9,9-dimethyl-10-(2-methyl-6-phenylpyridin-3-yl)-N²,N⁷-di(pyridin-2-yl)-9,10-dihydroacridine-2,7-diamine (272 mg, 0.48 mmol), 2-bromo pyridine (0.13 mL, 1.4 mmol), tris(dibenzylideneacetone) dipalladium (9.0 mg, 0.010 mmol) 2,2′-bis(diphenylphosphino)-1,1′-binaphthyl (BINAP) (13 mg, 0.021 mmol), and sodium t-butoxide (186 mg, 1.94 mmol) under a N₂ atmosphere was added toluene (40 mL) at room temperature. The flask was attached to a reflux condenser and heated to reflux. After 24 h, the crude reaction mixture was cooled to rt, diluted with water, and extracted with ethyl acetate. The organic layer was washed with water and brine, and then dried over MgSO₄. The resulting solution was filtered and concentrated. The resulting residue was purified via silica gel chromatography, eluted with ethyl acetate, to afford the product as a pale yellow solid in 86% yield. The material was recrystallized from hot methanol to afford an off-white solid.

¹H NMR (400 MHz, CDCl₃) δ 8.31 (ddd, J=4.9, 2.0, 0.9 Hz, 4H), 8.12-8.05 (m, 2H), 7.81 (d, J=8.2 Hz, 1H), 6.67 (d, J=8.2 Hz, 1H), 7.58-7.40 (m, 7H), 7.27 (d, J=2.4 Hz, 2H), 6.91 (dt, J=8.4, 0.8 Hz, 4H), 6.89 (ddd, J=7.2, 4.8, 0.8 Hz, 4H), 6.83 (dd, J=8.8, 2.4 Hz, 2H), 6.20 (d, J=8.7 Hz, 2H), 2.50 (s, 3H), 1.58 (s, 6H).

### Synthesis of EM-45

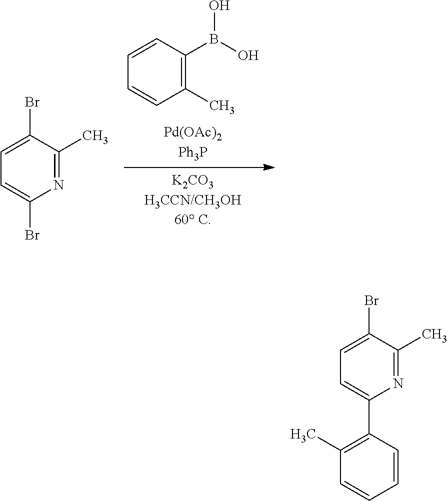

### 3-Bromo-2-methyl-6-(o-tolyl)pyridine

To a flask charged with 3,6-dibromo-2-methylpyridine (2.50 g, 9.96 mmol), 2-methyl phenyl boronic acid (1.59 g, 11.7 mmol), palladium acetate (112 mg, 0.498 mmol), triphenylphosphine (261 mg, 0.996 mmol), and potassium carbonate (2.75 g, 19.9 mmol) under a N₂ atmosphere was added acetonitrile (75 mL) and methanol (38 mL) at room temperature. The flask was attached to a reflux condenser and heated to 60° C. After 24 h, the crude reaction mixture was cooled to rt, and the volatiles were removed under reduced pressure. The resulting residue was dissolved in dichloromethane, and the organic layer was washed with water and brine. The washed organic layer was dried over Na₂SO₄, filtered and concentrated. The resulting residue was purified via silica gel chromatography, eluted with hexane:CH₂Cl₂=5:1, to afford the product as a white solid in 52% yield.

¹H NMR (400 MHz, CDCl₃) δ 7.85 (d, J=8.1 Hz, 1H), 7.39-7.34 (m, 1H), 7.24-7.238 (m, 3H), 7.85 (dd, J=8.2, 0.7 Hz, 1H), 2.72 (s, 3H), 2.36 (s, 3H).

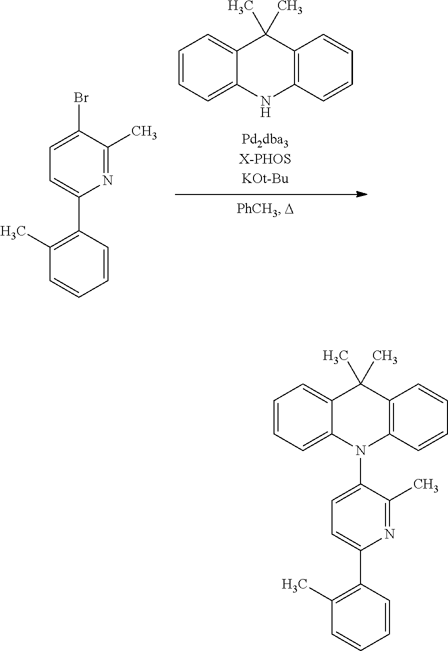

### 9,9-Dimethyl-10-(2-methyl-6-(o-tolyl)pyridin-3-yl)-9,10-dihydroacridine

To a flask charged with 3-bromo-2-methyl-6-(o-tolyl)pyridine (1.88 g, 7.17 mmol), 9,9-dimethyl-9,10-dihydroacridine (1.25 g, 5.97 mmol), tris(dibenzylideneacetone) dipalladium (109 mg, 0.119 mmol), 2-(dicyclohexylphosphino)-2′,4′,6′-tri-i-propyl-1,1′-biphenyl (X-PHOS) (171 mg, 0.358 mmol), and potassium t-butoxide (1.34 g, 11.9 mmol) under a N₂ atmosphere was added toluene (50 mL) at room temperature. The flask was attached to a reflux condenser and heated to reflux. After 24 h, the crude reaction mixture was cooled to rt, diluted with water, and extracted with ethyl acetate. The organic layer was washed with water and brine, and then dried over MgSO₄. The resulting solution was filtered and concentrated. The resulting residue was purified via silica gel chromatography, eluted with hexane:CH₂Cl₂=1:1, to afford the product as a white solid in 73% yield.

¹H NMR (400 MHz, CDCl₃) δ 7.64 (d, J=8.0 Hz, 1H), 7.56 (ddd, J=6.2, 3.2, 1.8 Hz, 1H), 7.53-7.46 (m, 3H), 7.40-7.29 (m, 3H), 7.08-6.91 (m, 4H), 6.17 (dd, J=8.1, 1.4 Hz, 2H), 2.49 (s, 3H), 2.39 (s, 3H), 1.76 (s, 3H), 1.69 (s, 3H).

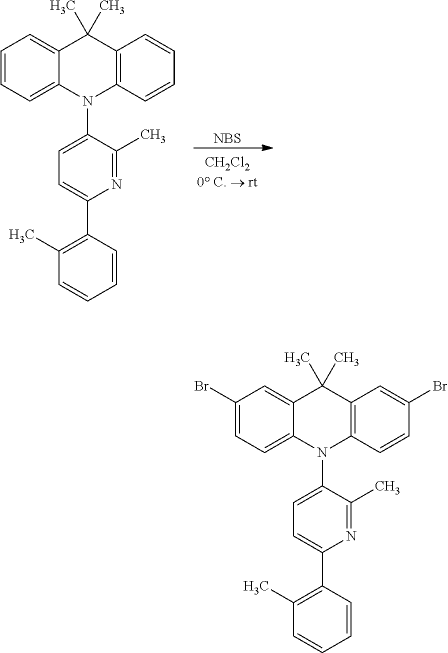 [SMILES: Cc1ccccc1-c1ccc(N2c3ccccc3C(C)(C)c3ccccc32)c(C)n1 ; "NBS" ; Cc1ccccc1-c1ccc(N2c3ccc(Br)cc3C(C)(C)c3cc(Br)ccc32)c(C)n1]

### 2,7-dibromo-9,9-dimethyl-10-(2-methyl-6-(o-tolyl)pyridin-3-yl)-9,10-dihydroacridine

To a flask charged with 9,9-dimethyl-10-(2-methyl-6-(o-tolyl)pyridin-3-yl)-9,10-dihydroacridine (1.71 g, 4.38 mmol) was added dichoromethane (70 mL) at room temperature. The flask was cooled to 0° C. and N-bromosuccinimide (1.64 g, 9.20 mmol) was added in 5 portions over 10 min. The reaction was stirred at 0° C. for 1 h, and then stirred at rt for 2 h. The crude reaction mixture was diluted with water, and extracted with CH₂Cl₂. The combined organic layers were washed with water and brine, and then dried over Na₂SO₄. The resulting solution was filtered and concentrated. The resulting residue was purified via silica gel chromatography, eluted with hexane:CH₂Cl₂=1:1, to afford the product as a white solid in 94% yield.

¹H NMR (400 MHz, CDCl₃) δ 7.64-7.48 (m, 5H), 7.40-7.29 (m, 3H), 7.12 (dd, J=8.8, 2.3 Hz, 2H), 6.05 (d, J=8.8 Hz, 2H), 2.48 (s, 3H), 2.34 (s, 3H), 1.71 (s, 3H), 1.66 (s, 3H).

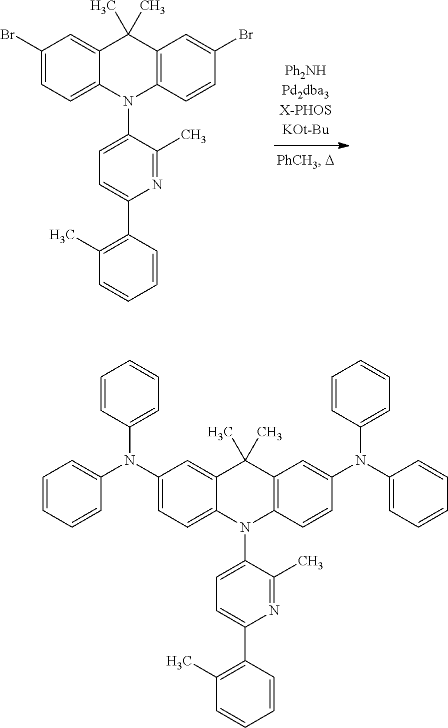 [SMILES: Cc1ccccc1-c1ccc(N2c3ccc(Br)cc3C(C)(C)c3cc(Br)ccc32)c(C)n1 ; Cc1ccccc1-c1ccc(N2c3ccc(N(c4ccccc4)c4ccccc4)cc3C(C)(C)c3cc(N(c4ccccc4)c4ccccc4)ccc32)c(C)n1]

### 9,9-Dimethyl-10-(2-methyl-6-(o-tolyl)pyridin-3-yl)-N²,N²,N⁷,N⁷-tetraphenyl-9,10-dihydroacridine-2,7-diamine (EM-45)

To a flask charged with 2,7-dibromo-9,9-dimethyl-10-(2-methyl-6-(o-tolyl)pyridin-3-yl)-9,10-dihydroacridine (2.25 g, 4.10 mmol), diphenylamine (1.67 g, 9.85 mmol), tris(dibenzylideneacetone) dipalladium (150 mg, 0.164 mmol), 2-(dicyclohexylphosphino)-2′,4′,6′-tri-i-propyl-1,1′-biphenyl (X-PHOS) (235 mg, 0.492 mmol), and potassium t-butoxide (1.84 g, 16.4 mmol) under a N₂ atmosphere was added toluene (55 mL) at room temperature. The flask was attached to a reflux condenser and heated to reflux. After 24 h, the crude reaction mixture was cooled to rt, diluted with water, and extracted with ethyl acetate. The organic layer was washed with water and brine, and then dried over MgSO₄. The resulting solution was filtered and concentrated. The resulting residue was purified via silica gel chromatography, eluted with hexane:CH₂Cl₂=1:2, to afford the product as a pale yellow solid in 83% yield. The material was recrystallized from hot methanol to afford an off-white solid.

¹H NMR (400 MHz, C₆D₆) δ 7.67-7.60 (m, 1H), 7.45 (d, J=2.5 Hz, 2H), 7.22-7.09 (m, 21H), 7.09-7.00 (m, 9H), 6.81 (tt, J=7.2, 1.2 Hz, 4H), 6.70 (dd, J=8.8, 2.5 Hz, 2H), 6.12 (d, J=8.8 Hz, 2H), 2.48 (s, 3H), 2.46 (s, 3H), 1.40 (br s, 6H).

### Synthesis of EM-46

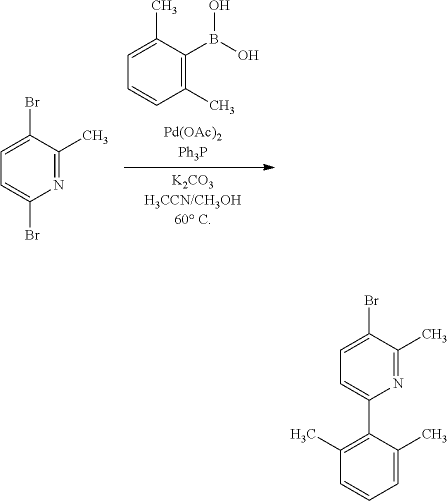

3-Bromo-6-(2,6-dimethylphenyl)-2-methylpyridine. To a flask charged with 3,6-dibromo-2-methylpyridine (2.50 g, 9.96 mmol), 2,6-dimethylphenyl boronic acid (1.75 g, 11.7 mmol), palladium acetate (112 mg, 0.498 mmol), triphenylphosphine (261 mg, 0.996 mmol), and potassium carbonate (2.75 g, 19.9 mmol) under a N₂ atmosphere was added acetonitrile (75 mL) and methanol (38 mL) at room temperature. The flask was attached to a reflux condenser and heated to 60° C. After 24 h, the crude reaction mixture was cooled to rt, and the volatiles were removed under reduced pressure. The resulting residue was dissolved in dichloromethane, and the organic layer was washed with water and brine. The washed organic layer was dried over Na₂SO₄, filtered and concentrated. The resulting residue was purified via silica gel chromatography, eluted with hexane:CH₂Cl₂=5:1, to afford the product as a white solid in 43% yield.

¹H NMR (400 MHz, CDCl₃) δ 7.87 (d, J=8.1 Hz, 1H), 7.18 (dd, J=8.3, 6.7 Hz, 1H), 7.09 (d, J=7.9 Hz, 2H), 6.94 (dd, J=8.0, 0.8 Hz, 1H), 2.72 (s, 3H), 2.05 (s, 6H).

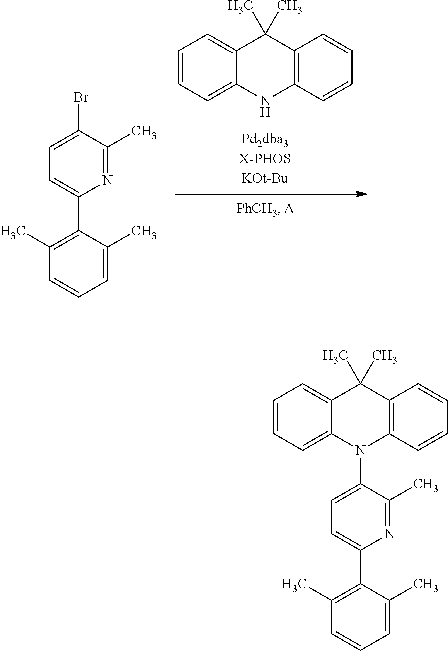

### 10-(6-(2,6-Dimethylphenyl)-2-methylpyridin-3-yl)-9,9-dimethyl-9-9,10-dihydroacridine

To a flask charged with 3-bromo-6-(2,6-dimethylphenyl)-2-methylpyridine (1.18 g, 4.30 mmol), 9,9-dimethyl-9,10-dihydroacridine (0.750 g, 3.58 mmol), tris(dibenzylideneacetone) dipalladium (66 mg, 0.072 mmol), 2-(dicyclohexylphosphino)-2′,4′,6′-tri-i-propyl-1,1′-biphenyl (X-PHOS) (103 mg, 0.215 mmol), and potassium t-butoxide (0.804 g, 7.17 mmol) under a N₂ atmosphere was added toluene (30 mL) at room temperature. The flask was attached to a reflux condenser and heated to reflux. After 24 h, the crude reaction mixture was cooled to rt, diluted with water, and extracted with ethyl acetate. The organic layer was washed with water and brine, and then dried over MgSO₄. The resulting solution was filtered and concentrated. The resulting residue was purified via silica gel chromatography, eluted with hexane:CH₂Cl₂=1:1, to afford the product as a white solid in 72% yield.

¹H NMR (400 MHz, CDCl₃) δ 7.66 (d, J=7.9 Hz, 1H), 7.51 (dd, J=7.6, 1.7 Hz, 2H), 7.33 (dd, J=7.9, 0.8 Hz, 1H), 7.25-7.20 (m, 1H), 7.16 (dd, J=7.1, 1.1 Hz, 2H), 7.04 (dd, J=8.1, 1.6 Hz, 2H), 6.98 (dt, J=7.4, 1.3 Hz, 2H), 6.15 (dd, J=8.1, 1.3 Hz, 2H), 2.38 (s, 3H), 2.20 (s, 6H), 1.76 (s, 3H), 1.67 (s, 3H).

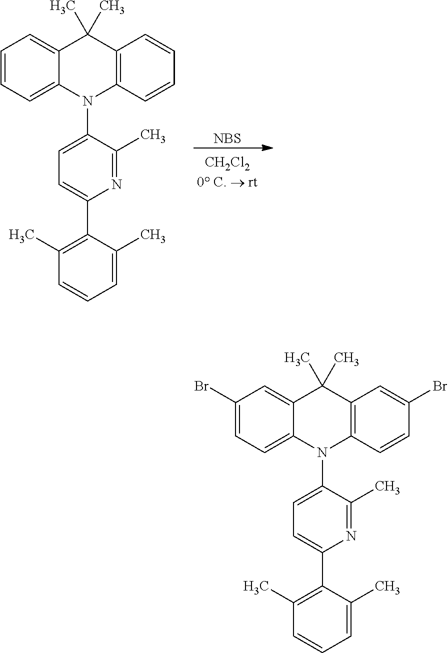 [SMILES: Cc1cccc(C)c1-c1ccc(N2c3ccccc3C(C)(C)c3ccccc32)c(C)n1 ; "NBS" ; Cc1cccc(C)c1-c1ccc(N2c3ccc(Br)cc3C(C)(C)c3cc(Br)ccc32)c(C)n1]

### 2,7-Dibromo-10-(6-(2,6-dimethylphenyl)-2-methylpyridin-3-yl)-9,9-dimethyl-9,10-dihydroacridine

To a flask charged with 10-(6-(2,6-dimethylphenyl)-2-methylpyridin-3-yl)-9,9-dimethyl-9,10-dihydroacridine (1.04 g, 2.57 mmol) was added dichoromethane (40 mL) at room temperature. The flask was cooled to 0° C. and N-bromosuccinimide (0.961 g, 5.40 mmol) was added in 5 portions over 10 min. The reaction was stirred at 0° C. for 1 h, and then stirred at rt for 2 h. The crude reaction mixture was diluted with water, and extracted with CH₂Cl₂. The combined organic layers were washed with water and brine, and then dried over Na₂SO₄. The resulting solution was filtered and concentrated. The resulting residue was purified via silica gel chromatography, eluted with hexane:CH₂Cl₂=1:1, to afford the product as a white solid in 89% yield.

¹H NMR (400 MHz, CDCl₃) δ 7.62 (d, J=7.9 Hz, 1H), 7.57 (d, J=2.3 Hz, 1H), 7.35 (d, J=7.9 Hz, 1H), 7.25-7.22 (m, 1H), 7.19-7.12 (m, 4H), 6.01 (d, J=8.8 Hz, 2H), 2.34 (s, 3H), 2.17 (s, 6H), 1.71 (s, 3H), 1.67 (s, 3H).

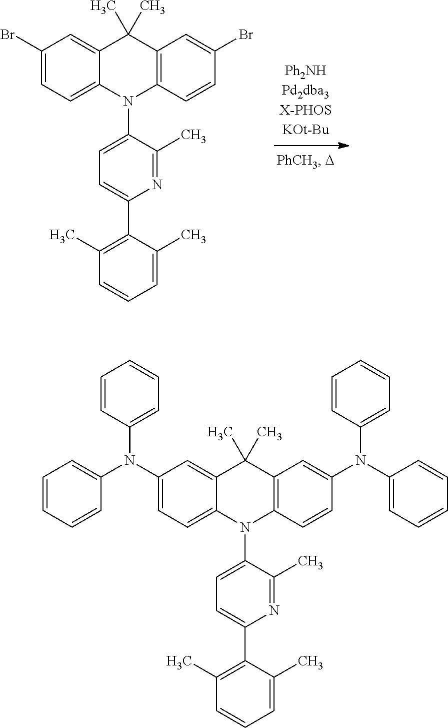 [SMILES: Cc1cccc(C)c1-c1ccc(N2c3ccc(Br)cc3C(C)(C)c3cc(Br)ccc32)c(C)n1 ; Cc1cccc(C)c1-c1ccc(N2c3ccc(N(c4ccccc4)c4ccccc4)cc3C(C)(C)c3cc(N(c4ccccc4)c4ccccc4)ccc32)c(C)n1]

### 10-(6-(2,6-Dimethylphenyl)-2-methylpyridin-3-yl)-9,9-dimethyl-N²,N²,N⁷,N⁷-tetraphenyl-9,10-dihydroacridine-2,7-diamine (EM-46)

To a flask charged with 2,7-dibromo-10-(6-(2,6-dimethylphenyl)-2-methylpyridin-3-yl)-9,9-dimethyl-9,10-dihydroacridine (1.29 g, 2.29 mmol), diphenylamine (0.932 g, 5.51 mmol), tris(dibenzylideneacetone) dipalladium (84 mg, 0.092 mmol), 2-(dicyclohexylphosphino)-2′,4′,6′-tri-i-propyl-1,1′-biphenyl (X-PHOS) (131 mg, 0.275 mmol), and potassium t-butoxide (1.03 g, 9.18 mmol) under a N₂ atmosphere was added toluene (30 mL) at room temperature. The flask was attached to a reflux condenser and heated to reflux. After 24 h, the crude reaction mixture was cooled to rt, diluted with water, and extracted with ethyl acetate. The organic layer was washed with water and brine, and then dried over MgSO₄. The resulting solution was filtered and concentrated. The resulting residue was purified via silica gel chromatography, eluted with hexane:CH₂Cl₂=1:2, to afford the product as a pale yellow solid in 71% yield. The material was recrystallized from hot methanol to afford an off-white solid.

¹H NMR (400 MHz, C₆D₆) 67.45 (d, J=2.5 Hz, 2H), 7.22-7.07 (m, 10H), 7.07-7.02 (m, 10H), 6.84 (tt, J=7.2, 1.2 Hz, 4H), 6.76 (d, J=7.6 Hz, 1H), 6.73 (dd, J=8.8, 2.8 Hz. 2H), 6.19 (d, J=8.8 Hz, 2H), 2.45 (s, 3H), 2.16 (s, 6H), 1.40 (s, 6H).

### Synthesis of EM-09

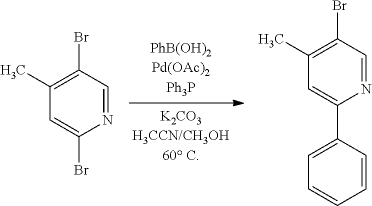

### 5-Bromo-4-methyl-2-phenylpyridine

To a flask charged with 2,5-dibromo-4-methylpyridine (4.69 g, 18.7 mmol), phenyl boronic acid (2.74 g, 22.4 mmol), palladium acetate (210 mg, 0.935 mmol), triphenylphosphine (490 mg, 1.87 mmol), and potassium carbonate (5.17 g, 37.4 mmol) under a N₂ atmosphere was added acetonitrile (130 mL) and methanol (65 mL) at room temperature. The flask was attached to a reflux condenser and heated to 60° C. After 24 h, the crude reaction mixture was cooled to rt, and the volatiles were removed under reduced pressure. The resulting residue was dissolved in dichloromethane, and the organic layer was washed with water and brine. The washed organic layer was dried over Na₂SO₄, filtered and concentrated. The resulting residue was purified via silica gel chromatography, eluted with hexane:CH₂Cl₂=5:1, to afford the product as a white solid in 43% yield.

¹H NMR (400 MHz, CDCl₃) δ 8.70 (s, 1H), 7.98-7.92 (m, 2H), 7.59 (s, 1H), 7.51-7.38 (m, 3H), 2.46 (s, 3H).

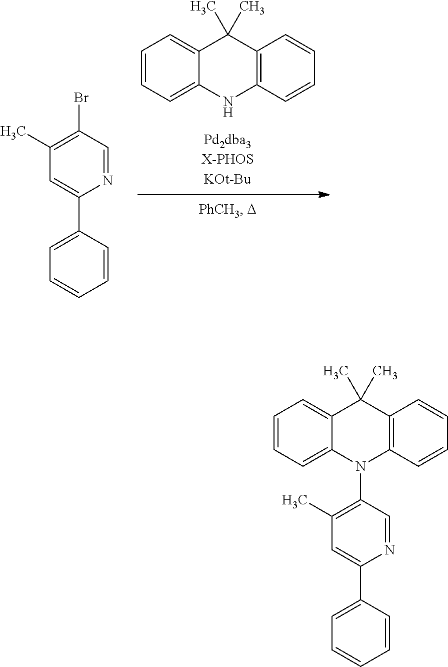 [SMILES: Cc1cc(-c2ccccc2)ncc1Br ; CC1(C)c2ccccc2Nc2ccccc21 ; Cc1cc(-c2ccccc2)ncc1N1c2ccccc2C(C)(C)c2ccccc21]

### 9,9-Dimethyl-10-(4-methyl-6-phenylpyridin-3-yl)-9,10-dihydroacridine

To a flask charged with 5-bromo-4-methyl-2-phenylpyridine (1.99 g, 8.03 mmol), 9,9-dimethyl-9,10-dihydroacridine (1.40 g, 6.69 mmol), tris(dibenzylideneacetone) dipalladium (122 mg, 0.134 mmol), 2-(dicyclohexylphosphino)-2′,4′,6′-tri-i-propyl-1,1′-biphenyl (X-PHOS) (191 mg, 0.401 mmol), and potassium t-butoxide (1.50 g, 13.4 mmol) under a N₂ atmosphere was added toluene (55 mL) at room temperature. The flask was attached to a reflux condenser and heated to reflux. After 24 h, the crude reaction mixture was cooled to rt, diluted with water, and extracted with ethyl acetate. The organic layer was washed with water and brine, and then dried over MgSO₄. The resulting solution was filtered and concentrated. The resulting residue was purified via silica gel chromatography, eluted with hexane:CH₂Cl₂=1:1, to afford the product as a white solid in 58% yield.

¹H NMR (400 MHz, CDCl₃) δ 8.52 (s, 1H), 8.14-8.06 (m, 2H), 7.86 (s, 1H), 7.57-7.43 (m, 5H), 7.02-6.93 (m, 4H), 6.20 (dd, J=8.0, 1.4 Hz, 2H), 2.17 (s, 3H), 1.75 (s, 3H), 1.71 (s, 3H).

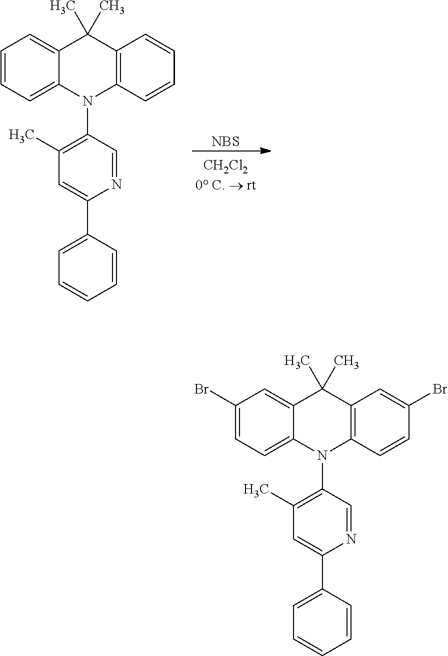 [SMILES: Cc1cc(-c2ccccc2)ncc1N1c2ccccc2C(C)(C)c2ccccc21 ; "NBS" ; Cc1cc(-c2ccccc2)ncc1N1c2ccc(Br)cc2C(C)(C)c2cc(Br)ccc21]

### 2,7-Dibromo-9,9-dimethyl-10-(4-methyl-6-phenylpyridin-3-yl)-9,10-dihydroacridine

To a flask charged with 9,9-dimethyl-10-(4-methyl-6-phenylpyridin-3-yl)-9, 10-dihydroacridine (1.47 g, 3.90 mmol) was added dichoromethane (60 mL) at room temperature. The flask was cooled to 0° C. and N-bromosuccinimide (1.46 g, 8.18 mmol) was added in 5 portions over 10 min. The reaction was stirred at 0° C. for 1 h, and then stirred at rt for 2 h. The crude reaction mixture was diluted with water, and extracted with CH₂Cl₂. The combined organic layers were washed with water and brine, and then dried over Na₂SO₄. The resulting solution was filtered and concentrated. The resulting residue was purified via silica gel chromatography, eluted with hexane:CH₂Cl₂=1:1, to afford the product as a white solid in 84% yield.

¹H NMR (400 MHz, CDCl₃) δ 8.48 (s, 1H), 8.13-8.05 (m, 2H), 7.87 (s, 1H), 7.60-7.44 (m, 5H), 7.09 (dd, J=8.8, 2.3 Hz, 2H), 6.07 (d, J=8.8 Hz, 2H), 2.14 (s, 3H), 1.70 (s, 3H), 1.67 (s, 3H).

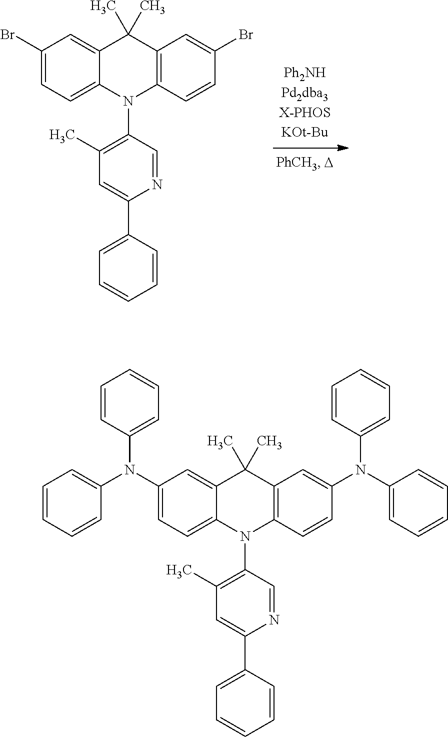 [SMILES: Cc1cc(-c2ccccc2)ncc1N1c2ccc(Br)cc2C(C)(C)c2cc(Br)ccc21 ; Cc1cc(-c2ccccc2)ncc1N1c2ccc(N(c3ccccc3)c3ccccc3)cc2C(C)(C)c2cc(N(c3ccccc3)c3ccccc3)ccc21]

### 9,9-Dimethyl-10-(4-methyl-6-phenylpyridin-3-yl)-N²,N²,N⁷,N⁷-tetraphenyl-9,10-dihydroacridine-2,7-diamine (EM-09)

To a flask charged with 2,7-dibromo-9,9-dimethyl-10-(4-methyl-6-phenylpyridin-3-yl)-9,10-dihydroacridine (1.74 g, 3.24 mmol), diphenylamine (1.32 g, 7.79 mmol), tris(dibenzylideneacetone) dipalladium (119 mg, 0.130 mmol), 2-(dicyclohexylphosphino)-2′,4′,6′-tri-i-propyl-1,1′-biphenyl (X-PHOS) (186 mg, 0.389 mmol), and potassium t-butoxide (1.46 g, 13.0 mmol) under a N₂ atmosphere was added toluene (50 mL) at room temperature. The flask was attached to a reflux condenser and heated to reflux. After 24 h, the crude reaction mixture was cooled to rt, diluted with water, and extracted with ethyl acetate. The organic layer was washed with water and brine, and then dried over MgSO₄. The resulting solution was filtered and concentrated. The resulting residue was purified via silica gel chromatography, eluted with hexane:CH₂Cl₂=1:2, to afford the product as a pale yellow solid in 86% yield. The material was recrystallized from hot methanol to afford an off-white solid.

¹H NMR (400 MHz, acetone-d₆) δ 8.56 (s, 1H), 8.28-8.20 (m, 2H), 8.17 (s, 1H), 7.58-7.44 (m, 3H), 7.33 (d, J=2.5 Hz, 2H), 7.30-7.20 (m, 8H), 7.07-7.00 (m, 8H), 6.99-6.94 (m, 4H), 6.81 (dd, J=8.8, 2.5 Hz, 2H), 6.24 (d, J=8.7 Hz, 2H), 2.26 (s, 3H), 1.56 (s, 6H).

Computational Evaluation:

The ground-state (S₀) and first excited triplet-state (T₁) configurations of the molecules were computed using Density Functional Theory (DFT) at B3LYP/6-31g* level. The energies of highest occupied molecular orbital (HOMO) and lowest unoccupied molecular orbital (LUMO) were obtained from the S₀ configuration. The energy of the T₁ state was computed as the difference in energy between the minima of S₀ and T₁ potential energy surfaces (PES). The S₁-T₁ gap was computed as the vertical energy between the S₁ and T₁ states, at the T₁ configuration. The S₁-T₁ gap was computed using Time Dependent Density Functional Theory (TDDFT). All the calculations were performed using G09 suit of programs.

TABLE 1
Summary of Computational Data for EM-01-EM-517.

| Emitter | HOMO (eV) | LUMO (eV) | Triplet (eV) | S₁-T₁gap (eV) |
|---|---|---|---|---|
| EM-01 | −4.93 | −1.65 | 2.63 | 0.16 |
| EM-06 | −4.46 | −1.40 | 2.60 | 0.32 |
| EM-07 | −4.38 | −1.46 | 2.45 | 0.34 |
| EM-08 | −4.46 | 1.31 | 2.67 | 0.26 |
| EM-09 | −4.47 | −1.31 | 2.70 | 0.25 |
| EM-23 | −4.69 | −1.22 | 2.75 | 0.37 |
| EM-45 | −4.46 | −1.15 | 2.79 | 0.05 |
| EM-46 | −4.47 | −0.96 | 2.82 | 0.21 |
| EM-54 | −4.35 | −1.41 | 2.72 | 0.01 |
| EM-64 | −4.89 | −1.56 | 2.70 | 0.42 |
| EM-76 | −4.65 | −1.48 | 2.76 | 0.04 |
| EM-241 | −4.38 | −1.32 | 2.70 | 0.20 |
| EM-249 | −4.86 | −1.40 | 2.78 | 0.38 |
| EM-257 | −5.02 | −1.45 | 2.79 | 0.47 |
| EM-259 | −5.09 | −1.55 | 2.79 | 0.44 |
| EM-260 | −5.09 | −1.54 | 2.79 | 0.44 |
| EM-261 | −4.84 | −1.43 | 2.76 | 0.37 |
| EM-294 | −4.49 | −1.18 | 2.80 | 0.07 |
| EM-297 | −5.05 | −1.16 | 3.05 | 0.56 |
| EM-324 | −5.03 | −1.10 | 3.05 | 0.60 |
| EM-325 | −4.32 | −0.88 | 2.81 | 0.21 |
| EM-368 | −4.53 | −1.44 | 2.68 | 0.14 |
| EM-383 | −4.94 | −1.31 | 3.01 | 0.25 |
| EM-384 | −4.54 | −1.30 | 2.80 | 0.04 |
| EM-390 | −4.82 | −1.41 | 2.82 | 0.11 |
| EM-391 | −4.78 | −1.30 | 2.84 | 0.19 |
| EM-396 | −4.38 | −1.33 | 2.61 | 0.29 |
| EM-411 | −4.48 | −1.30 | 2.75 | 0.03 |
| EM-417 | −4.63 | −1.51 | 2.69 | 0.02 |
| EM-515 | −4.83 | −1.47 | 2.83 | 0.53 |
| EM-516 | −4.61 | −1.53 | 2.79 | 0.21 |
| EM-517 | −4.36 | −1.27 | 2.64 | 0.25 |

### Film Preparation and Photoluminescence Characterization:

### Measurement of Emission Properties

Emitter-doped polymer films utilized for photoluminescence spectroscopy were prepared by dissolving poly(methyl methacrylate) (PMMA) and the respective emitter in CH₂Cl₂. The PMMA/emitter complex mixtures were filtered through 45 μm PTFE filters and drop cast onto glass microscope coverslips. The resulting films were dried for 15 hours. They were then dried at 60° C., in a vacuum oven, at approximately 1×10⁻² torr (1.33 Pa), for several hours.

Room temperature and 77 K spectra reported herein are steady-state emission profiles collected on polymer films inside the sample chamber of a PTI fluorimeter. The profiles were collected using an excitation wavelength of 355 nm. The films were contained in standard borosilicate NMR tubes that were placed into quartz tipped EPR Dewars. Both room temperature and low temperature spectra were acquired in this manner. The low temperature spectra were acquired upon filling the Dewar with liquid nitrogen.

Time-resolved emission spectra were acquired on the same samples utilizing the pulsed capabilities of the PTI instrument. The experimental estimate for the S₁-T₁ gap is obtained by collecting time-resolved emission spectra for doped PMMA films of the inventive composition. Triplet energy level (T₁) is defined as the energy difference between the ground state singlet and lowest energy triplet excited state. This value is experimentally estimated by the x-axis intersection point of a tangent line drawn on the high energy side of the delayed component of the emission spectrum taken at 77 Kelvin (K). In cases where time-resolved spectra cannot be measured, the lowest energy peak at 77 Kelvin is used. The singlet energy level (S₁) is defined by the energy difference between the ground state singlet energy and the lowest energy singlet excited state. This value is experimentally estimated by the x-axis intersection point of a tangent line drawn on the high energy side of the prompt portion of the emission spectrum at 77 K. The S₁-T₁ gap is obtained by subtracting the S₁ and T₁ values.

TABLE 2
Summary of Experimental PL Characteristics
for EM-01-EM-64 in PMMA.

| | Room Temp | | |
|---|---|---|---|
| | Emission Max | Triplet | S₁-T₁ gap |
| Emitter | (nm) | (eV) | (eV) |
| EM-01 | 470 | 2.7 | 0.26 |
| EM-06 | 496 | 2.7 | 0.07 |
| EM-08 | 496 | 2.8 | 0.03 |
| EM-09 | 490 | 2.8 | 0.03 |
| EM-23 | 472 | 2.9 | 0.2 |
| EM-45 | 477 | 2.8 | 0.16 |
| EM-46 | 468 | 2.9 | 0.12 |
| EM-54 | 494 | 2.8 | 0.05 |
| EM-64 | 465 | 2.8 | 0.13 |

### OLED Device Fabrication and Testing

All organic materials were purified by sublimation before deposition. OLEDs were fabricated onto an ITO coated glass substrate that served as the anode, and topped with an aluminum cathode. All organic layers were thermally deposited by chemical vapor deposition, in a vacuum chamber with a base pressure of <10⁻⁷ torr. The deposition rates of organic layers were maintained at 0.1-0.05 nm/s. The aluminum cathode was deposited at 0.5 nm/s. The active area of the OLED device was “3 mm×3 mm,” as defined by the shadow mask for cathode deposition.

Each cell, containing HIL1, HIL2, HTL1, HTL2, EBL, EML host, EML dopant, ETL1, ETL2, or EIL, was placed inside a vacuum chamber, until it reached 10⁻⁶ torr. To evaporate each material, a controlled current was applied to the cell, containing the material, to raise the temperature of the cell. An adequate temperature was applied to keep the evaporation rate of the materials constant throughout the evaporation process.

For the HIL1 layer, N⁴,N⁴′-diphenyl-N⁴,N⁴′-bis(9-phenyl-9H-carbazol-3-yl)-[1,1′-biphenyl]-4,4′-diamine was evaporated at a constant 1 A/s rate, until the thickness of the layer reached 600 Angstrom. Simultaneously, the dipyrazino[2,3-f:2′,3′-h]quinoxaline-2,3,6,7,10,11-hexacarbonitrile layer was evaporated at a constant 0.5 A/s rate, until the thickness reached 50 Angstrom. For the HTL1 layer, N-([1,1′-biphenyl]-4-yl)-9,9-dimethyl-N-(4-(9-phenyl-9H-carbazol-3-yl)phenyl)-9H-fluoren-2-amine was evaporated at a constant 1 A/s rate, until the thickness reached 150 Angstrom. For the HTL2 layer, N,N-di([1,1′-biphenyl]-4-yl)-4′-(9H-carbazol-9-yl)-[1,1′-biphenyl]-4-amine was evaporated at a constant 1 A/s rate, until the thickness reached 50 Angstrom. For the EBL layer, 1,3-di(9H-carbazol-9-yl)benzene was evaporated at a constant 1 A/s rate, until the thickness reached 50 Angstrom. For the EML layer, 9,9′,9″-(pyrimidine-2,4,6-triyl)tris(9H-carbazole) (host) and 9,9-Dimethyl-N²,N²,N⁷,N⁷-tetraphenyl-10-(6-phenylpyridin-3-yl)-9,10-dihydroacridine-2,7-diamine (EM-06, dopant) or 9,9-dimethyl-10-(2-methyl-6-phenylpyridin-3-yl)-N²,N²,N⁷,N⁷-tetraphenyl-9,10-dihydroacridine-2,7-diamine (EM-08, dopant) were co-evaporated, until the thickness reached 400 Angstrom. The deposition rate for host material was 0.85 A/s, and the deposition for the dopant material was 0.15 A/s, resulting in a 15% doping of the host material. For the ETL1 layer, 5-(4-([1,1′-biphenyl]-3-yl)-6-phenyl-1,3,5-triazin-2-yl)-7,7-diphenyl-5,7-dihydroindeno[2,1-b]carbazole was evaporated at a constant 1 A/s rate, until the thickness reached 50 Angstrom. For the ETL2 layer, 2,4-bis(9,9-dimethyl-9H-fluoren-2-yl)-6-(naphthalen-2-yl)-1,3,5-triazine was co-evaporated with lithium quinolate(Liq), until the thickness reached 300 Angstrom. The evaporation rate for the ETL compounds and Liq was 0.5 A/s. Finally, “20 Angstrom” of a thin electron injection layer (Liq) was evaporated at a 0.5 A/s rate. See Table 2.

The current-voltage-brightness (J-V-L) characterizations for the OLED devices were performed with a source measurement unit (KEITHLY 238) and a luminescence meter (MINOLTA CS-100A). EL spectra of the OLED devices were collected by a calibrated CCD spectrograph and EQE was collected by a PR655.

TABLE 3
Device Materials

| | Name |
|---|---|
| Hole Injection | N⁴,N⁴′-diphenyl-N⁴,N⁴′-bis(9-phenyl-9H-carbazol-3- |
| Material 1 | yl)-[1,1′-biphenyl]-4,4′-diamine |
| Hole Injection | dipyrazino[2,3-f:2′,3′-h]quinoxaline-2,3,6,7,10,11- |
| Material 2 | hexacarbonitrile |
| Hole Transporting | N-[1,1′-biphenyl]-4-yl)-9,9-dimethyl- N-(4-(9-phenyl- |
| Material1 | 9H-carbazol-3-yl)phenyl)-9H-fluoren-2-amine |
| Hole Transporting | N,N-di([1,1′-biphenyl]-4-yl)-4′-(9H-carbazol-9-yl)- |
| Material2 | [1,1′-biphenyl]-4-amine |
| Electron blocking | 1,3-di(9H-carbazol-9-yl)benzene |
| Material2 | |
| Host | 9,9′,9″-(pyrimidine-2,4,6-triyl)tris(9H-carbazole) |
| Dopant | 9,9-Dimethyl-N²,N²,N⁷,N⁷-tetraphenyl-10-(6- |
| | phenylpyridin-3-yl)-9,10-dihydroacridine-2,7-diamine |
| | (15 wt %) or 9,9-dimethyl-10-(2-methyl-6- |
| | phenylpyridin-3-yl)-N²,N²,N⁷,N⁷-tetraphenyl-9,10- |
| | dihydroacridine-2,7-diamine (10 wt %) |
| ETL1 | 5-(4-([1,1′-biphenyl]-3-yl)-6-phenyl-1,3,5-triazin-2-yl)- |
| | 7,7-diphenyl-5,7-dihydroindeno[2,1-b]carbazole |
| ETL2 | 2,4-bis(9,9-dimethyl-9H-fluoren-2-yl)-6-(naphthalen-2- |
| | yl)-1,3,5-triazine |
| Electron Injection | lithium quinolate |
| Material | |

TABLE 4
Device Results

| | Luminous Efficiency | | |
|---|---|---|---|
| Emitter | @1000 nit [Cd/A] | CIE (X, Y) | EQE (%) |
| EM-06 | 19.1 | 268,470 | 7.4 |
| EM-08 | 20.4 | 254,470 | 11.8 |

1. A compound having a tricyclic nucleus and having formula (I) 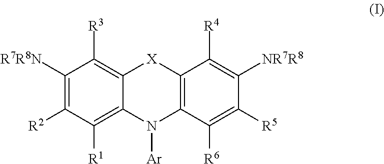
wherein Ar is C₃-C₂₅ aryl in which at least one aromatic ring atom is nitrogen; X is O, S or CR⁹R¹⁰; R¹, R², R³, R⁴, R⁵ and R⁶ independently are hydrogen, deuterium, C₁-C₁₂ alkyl, C₄-C₁₂ aryl or C₂-C₁₂ alkenyl; R⁷ and R⁸ independently are hydrogen, deuterium, C₁-C₁₂ alkyl, C₄-C₂₀ aryl or C₂-C₁₂ alkenyl or R⁷ and R⁸ groups attached to a nitrogen atom are joined by a single bond, O, S or CR¹¹R¹² to form a single nitrogen-containing substituent; R⁹ and R¹⁰ independently are hydrogen, deuterium, C₁-C₁₂ alkyl, C₄-C₁₂ aryl or C₂-C₁₂ alkenyl; and R¹¹ and R¹² independently are hydrogen, deuterium, C₁-C₁₂ alkyl, C₄-C₁₂ aryl or C₂-C₁₂ alkenyl; provided that Ar does not contain an aromatic ring attached to the tricyclic nucleus which contains more than two aromatic ring nitrogen atoms.

2. The compound of claim 1 in which R¹, R², R³, R⁴, R⁵ and R⁶ independently are hydrogen, deuterium, C₁-C₈ alkyl, C₄-C₁₂ aryl or C₂-C₈ alkenyl.

3. The compound of claim 2 in which X is O or CR⁹R¹⁰.

4. The compound of claim 3 in which Ar is C₆-C₂₀ aryl.

5. The compound of claim 4 in which R¹, R², R³, R⁴, R⁵ and R⁶ independently are hydrogen, deuterium or C₁-C₄ alkyl.

6. The compound of claim 5 in which no aromatic ring in Ar contains more than two aromatic ring nitrogen atoms.

7. The compound of claim 6 in which Ar has two or three aromatic rings.

8. The compound of claim 7 in which R⁹ and R¹⁰ independently are hydrogen, deuterium, C₆-C₁₀ aryl or C₁-C₄ alkyl; R¹¹ and R¹² independently are hydrogen, deuterium or C₁-C₄ alkyl; and R⁷ and R⁸ independently are C₄-C₁₅ aryl.

9. A light-emitting device comprising at least one compound of claim 1.

10. The light-emitting device of claim 9 in which said at least one compound is present in an emitter layer.
